(19) United States
(12) Patent Application Publication
(10) Pub. No.: US 2024/0081921 A1
(43) Pub. Date: Mar. 14, 2024

(54) SYSTEM AND METHOD FOR VERIFICATION OF CONVERSION OF LOCATIONS BETWEEN COORDINATE SYSTEMS

(71) Applicant: BEYEONICS SURGICAL LTD., Haifa (IL)

(72) Inventor: Rani BEN-YISHAI, Haifa (IL)

(73) Assignee: BEYEONICS SURGICAL LTD., Haifa (IL)

(21) Appl. No.: 18/518,463

(22) Filed: Nov. 23, 2023

Related U.S. Application Data
(63) Continuation of application No. PCT/IL2022/050564, filed on May 26, 2022.
(60) Provisional application No. 63/193,295, filed on May 26, 2021.

(51) Int. Cl. A61B 34/20; A61B 34/10; A61B 90/00; G06T 7/00

(52) U.S. Cl. CPC A61B 34/20; A61B 34/10; A61B 90/37; G06T 7/0012; A61B 2034/102; A61B 2034/105; A61B 2034/2065; A61B 2090/3735; G06T 2207/10101; G06T 2207/30041; G06T 2207/30101

(57) ABSTRACT

Systems and methods for providing a verification symbol relating to the validity of a conversion of locations between a first image and a second image for surgery are provided. Providing the verification symbol involves receiving a selection of an image in the first image that corresponds to a physical element in a second image and that has a location in a first coordinate system, determining the location in a second coordinate system associated with the second image by employing a conversion of locations, and displaying a verification symbol superimposed with the second image based on the corresponding location in the second coordinate system.

### CROSS REFERENCE TO RELATED APPLICATIONS

This Application is a Continuation of PCT Application No. PCT/IL2022/050564 filed on May 26, 2022, claiming priority from U.S. Provisional Patent Application No. 63/193,295 filed on May 26, 2021, both of which are incorporated herein by reference in their entirety.

### FIELD OF THE INVENTION

The invention generally relates to verification of conversion between coordinate systems and more particularly, to methods and systems for providing verification symbols indicating the validity of a conversion between coordinate systems.

### BACKGROUND OF THE INVENTION

Registration of coordinate systems may be important in many fields of endeavor e.g., in medical imaging and/or tracking systems, when a registration of coordinate systems between two images, between 3D datasets and images, and/or between 3D datasets and tracking systems, is employed to represent in one coordinate system, an object (e.g., virtual or physical) present in another coordinate system.

For example, in some medical imaging systems a pre-operative image is registered with an intraoperative image. In another example, a representation of a medical tool tracked in a tracking coordinate system may be shown overlaid on an image derived from a 3D dataset (e.g., a CT scan, an MRI scan) of the region in which the object is tracked, and where the 3D dataset is associated with a respective coordinate system (e.g., a scan coordinate system). A transformation may be determined between the scan coordinate system and the tracking coordinate system, such that at least a portion of the points in the tracking coordinate system are associated with corresponding points in the scan coordinate system and vice versa. In another example, when an image of a model derived from a 3D dataset of a region of interest (e.g., by segmentation) is to be overlaid on an acquired image of that region, a transformation may be determined between the model coordinate system (e.g., the scan coordinate system) and the image coordinate system, such that at least a portion of the points in the image coordinate system are associated with corresponding points in the model coordinate system and vice versa.

One difficulty may be that registration of coordinates systems may be prone to errors. Another difficulty is that a valid registration of coordinates may become invalid over time due to, for example, an occurrence of an event. For example, a tracking reference unit in a tracking system may move (e.g., accidently or unintentionally). In another example, a body part that is being operated on and which is present in a model employed during the procedure, may move relative to another body part present in the model, and that was used for registration (e.g., brain shift during brain surgery). In such cases, an initially determined transformation between coordinate systems may be erroneous in the area being operated on.

Currently, methods and systems exist for presenting a line marker to a surgeon that may assist the surgeon in orienting an intraocular lens which is inserted into an eye of a patient, relative to the eye (e.g., a toric intraocular lens that is required to be correctly oriented). Current systems may include a surgical microscope system having two ocular beam paths, a camera, an image projector and/or a controller. A planned orientation of the intraocular lens may be predetermined with respect to a preoperative image. In some current systems, to display the line marker, a first semi-transparent mirror (e.g., a beam splitter) may be positioned in one beam path and direct light to the camera. In some current systems, the camera may acquire an intraoperative image of the eye and provide the acquired image to the controller. In some current systems, the controller may compare the intraoperative image and the preoperative image to, for example, determine a cyclorotation of the eye, and/or determine a location of the line marker with respect to the intraoperative image. In some current systems, the controller may generate an image of the line marker at the determined location, and the image projector projects the image toward the other ocular beam path. In some current systems, another semi-transparent mirror may project the image of the line marker toward the eye of the user, thus combining the image of the line marker with the view of the eye (e.g., as seen by the user). When the intraocular lens is correctly oriented in the eye according to its target orientation, axis marks of the intraocular lens may coincide with the line marker.

In some current systems, the user may rely on guidance that is displayed during the procedure, but the user may not know in real-time whether the overlay is reliable or not.

In some current systems, the user may discontinue the regular flow of the procedure and/or check the validity of the guidance. For instance, in some spine navigation systems, the user may stop the procedure to check the reliability of the guidance by pointing a tracked pointer at an anatomical element in the surgical field, and checking that a virtual representation of the pointer, that is overlaid on CT or MRI images displayed via a monitor, is correctly pointing at the representation of the anatomical element in the imaging dataset. This method for verifying the reliability of the guidance may be cumbersome, as it may interfere with the regular flow of the procedure. Also, it may provide a verification only for the moment it is performed and may not provide the surgeon with confidence regarding the reliability of the guidance continuously throughout the procedure.

### SUMMARY OF THE INVENTION

Advantages of the invention may include providing an indicator that may allow for visual verification of the validity of a conversion of locations between coordinate systems. Advantages of the invention may also include the verification of the validity of conversion of locations being done without discontinuing the regular workflow of the procedure.

In one aspect, the invention involves a method for providing a verification symbol relating to a validity of a conversion of locations between a first image of an eye of a patient and a second image of the eye of the patient, employed for ophthalmic surgery. The method may involve receiving a selection of an image element in the first image, the image element corresponding to a physical element in the second image, the image element having a location in a first coordinate system, the first coordinate system being associated with the first image. The method may involve determining for the location in the first coordinate system a corresponding location in a second coordinate system being associated with the second image by employing the conversion of locations between the first coordinate system and the second coordinate system. The method may involve displaying the verification symbol superimposed with the second image based on the corresponding location in the second coordinate system. In some embodiments, the selection of the image element is performed either manually or automatically.

In some embodiments, wherein the verification symbol is based on the image element, guidance information displayed with the second image, or any combination thereof. In some embodiments, wherein at least one of the first or second images is intraoperative.

In some embodiments, wherein the at least one image element is at least one of a scleral blood vessel, a retinal blood vessel, a bifurcation point, a contour of the limbus, and a visible element on the iris.

In some embodiments, the method involves displaying guidance information defined with respect to the first or second coordinate systems superimposed with one of the first image or the second image respectively, employing the conversion of locations.

In some embodiments, the guidance information comprises at least one of: information indicating a planned location and/or orientation of an intraocular lens, information indicating an actual location and/or orientation of an intraocular lens, information indicating a planned incision, information indicating a planned location and/or orientation of an implant for glaucoma, information relating to planned sub-retinal injection, information relating to a membrane removal, information indicating a location of an OCT scan, and information indicating a footprint of a field of view of an endoscope.

In another aspect, the invention includes a system for providing visual information relating to a validity of a conversion of locations between a first image and a second image, the first image and second image employed for ophthalmic surgery. The system includes a camera configured to acquire the first image, the second image or both. The system includes a processor, coupled with the camera configured to select an image element in the first image, the image element corresponding to a physical element in the second image, the image element having a location in a first coordinate system, the first coordinate system being associated with the first image. The processor may also be configured to determine for the location in the first coordinate system a corresponding location in a second coordinate system being associated with the second image by employing the conversion of locations between the first coordinate system and the second coordinate system. The processor may also be configured to display the verification symbol superimposed with the second image based on the corresponding location in the second coordinate system.

In some embodiments, the verification symbol is based on the image element, guidance information displayed with the second image, or any combination thereof.

In some embodiments, at least one of the first or second images is intraoperative. In some embodiments, the at least one image element is at least one of: a scleral blood vessel, a retinal blood vessel, a bifurcation point, a contour of the limbus, and a visible element on the iris.

In some embodiments, the processor is further configured to display guidance information defined with respect to one of the first or second coordinate systems superimposed with one of the first image or the second image respectively, employing the conversion of locations.

In some embodiments, the guidance information comprises at least one of: information indicating a planned location and/or orientation of an intraocular lens, information indicating an actual location and/or orientation of an intraocular lens, information indicating a planned incision, information indicating a planned location and/or orientation of an implant for glaucoma, information relating to planned sub-retinal injection, information relating to a membrane removal, information indicating a location of an OCT scan, and information indicating a footprint of a field of view of an endoscope.

In some embodiments, the guidance information is audio. In some embodiments, the first image is a two-dimensional intraoperative image and the second image is two dimensional intraoperative image with three-dimensional information displayed thereon.

In some embodiments, the verification symbol is further based on data related to the conversion of location.

In another aspect, the invention involves a method for providing a verification symbol relating to a validity of a conversion of locations between a first image of a patient and a second image the patient, employed for surgery. The method may involve receiving a selection of an image element in the first image, the image element corresponding to a physical element in the second image, the image element having a location in a first coordinate system, the first coordinate system being associated with the first image. The method may also involve determining for the location in the first coordinate system a corresponding location in a second coordinate system being associated with the second image by employing the conversion of locations between the first coordinate system and the second coordinate system. The method may also involve displaying the verification symbol superimposed with the second image based on the corresponding location in the second coordinate system.

In some embodiments, the first image and the second image are of at least a portion of a brain, at least a portion of a spine, at least a portion of a tumor to be treated, at least a portion of an eye, soft tissue or hard tissue.

In another aspect, the invention involves a method for providing a verification symbol relating to a validity of a conversion of locations between a first image and a second image, the first image and the second image employed for ophthalmic surgery. The method may involve receiving a first image associated with a first coordinate system. The method may involve receiving guidance information with respect to the first image. The method may involve receiving a second image associated with a second coordinate system, the second image representing an optical image of a scene. The method may involve receiving a selection of an image element in the first image, the image element corresponding to a physical element in the optical image, the image element having a first location in the first coordinate system. The method may involve determining for the first location in the first coordinate system a corresponding second location in the second coordinate system by employing the conversion of locations between the first coordinate system and the second coordinate system. The method may involve determining a third location in the second image of a guidance symbol generated based on the guidance information, by employing the conversion of locations from the first coordinate system to the second coordinate system. The method may involve generating an overlay image comprising the guidance symbol and the verification symbol based on the determined third and second locations, respectively. The method may involve displaying the overlay image superimposed with the optical image.

In some embodiments, the guidance information is received as a superimposition with the first image. In some embodiments, the guidance information is received separately from the first image.

In another aspect, the invention includes a system for providing a verification symbol relating to a validity of a conversion of locations between a first image and a second image, the first image and the second image employed for ophthalmic surgery. The system may include a camera configured to acquire the first image, the second image or both. The system may include a processor, coupled with the camera configured to: receive a first image associated with a first coordinate system, receive guidance information with respect to the first image, receive a second image associated with a second coordinate system, the second image representing an optical image of a scene. The processor may also be configured to select an image element in the first image, the image element corresponding to a physical element in the optical image, the image element having a first location in the first coordinate system, determine for the first location in the first coordinate system a corresponding second location in the second coordinate system by employing the conversion of locations between the first coordinate system and the second coordinate system, and determine a third location in the second image of a guidance symbol generated based on the guidance information, by employing the conversion of locations from the first coordinate system to the second coordinate system. The processor may also be configured to generate an overlay image comprising the guidance symbol and the verification symbol based on the determined third and second locations, respectively and displaying the overlay image superimposed with the optical image.

In some embodiments, the guidance information is received as a superimposition with the first image. In some embodiments, the guidance information is received separately from the first image.

In another aspect, the invention involves method for providing a verification symbol relating to a validity of an effective alignment of a tool tracking unit with a medical tool employed in a medical procedure. The method may involve determining a tool alignment, or receiving a predetermined tool alignment, between the medical tool and the tool tracking unit. The method may also involve receiving information relating to a geometry of the medical tool. The method may also involve generating the verification symbol based on the tool alignment and the information relating to the geometry of the medical tool.

In some embodiments, the method involves displaying the verification symbol superimposed with an image acquired by a camera. In some embodiments, the method involves displaying the verification symbol superimposed with an optical image. In some embodiments, when the tool tracking unit and the medical tool are effectively aligned, the verification symbol and the medical tool appear visually in alignment, and when the tool tracking unit and the medical tool are effectively misaligned, the verification symbol and the medical tool appear visually in misalignment.

In some embodiments, a source of the effective misalignment is one or more of: tool misalignment, deformation of the medical tool, and movement of an HWD relative to a head of a user.

In another aspect, the invention involves a method for determining alignment of a tool tracking unit with a medical tool employed in a medical procedure. The method may involve acquiring image information of the medical tool by a camera system. The method may involve determining position and orientation (P&O) of a tool tracking unit attached to the medical tool in a tracking coordinate system. The method may involve determining tool alignment between the medical tool and the tool tracking unit based on the acquired image information and the determined P&O of the tool tracking unit.

In another aspect, the invention involves a method for eye location calibration. The method may involve i) generating a tool verification symbol based on a current location of an eye relative to an HMD and ii) receiving adjustments to adjust xyz values of the current location of the relative to the HMD. The method may also involve repeating steps i) and ii) until the tool verification symbol is sufficiently aligned with the tool.

In some embodiments, the adjustments are received from a user via a user interface. In some embodiments, the user interface is a voice command, foot switch, or any combination thereof.

### BRIEF DESCRIPTION OF THE DRAWINGS

Non-limiting examples of embodiments of the disclosure are described below with reference to FIGS. attached hereto. Dimensions of features shown in the FIGS. are chosen for convenience and clarity of presentation and are not necessarily shown to scale. The subject matter regarded as the invention is particularly pointed out and distinctly claimed in the concluding portion of the specification. The invention, however, both as to organization and method of operation, together with objects, features, and advantages thereof, may be understood by reference to the following detailed description when read with the accompanied drawings. Embodiments are illustrated without limitation in the FIGS., in which like reference numerals indicate corresponding, analogous, or similar elements, and in which:

FIG. 1A is schematic block diagram of system 100 according to some embodiments of the invention;

FIG. 1B is a block diagram of a camera assembly of FIG. 1A, according to some embodiments of the invention;

FIG. 1C is a schematic illustration of an operating scenario of system of FIG. 1A, according to some embodiments of the invention;

FIGS. 2A, 2B, 2C and 2D are schematic diagrams of a preoperative image and an intraoperative image of an eye, during placement of a toric Intraocular Lens (IOL), according to some embodiments of the invention;

FIGS. 3A, 3B, 3C and 3D are schematic diagrams of a preoperative image and an intraoperative image of an eye, during placement of a toric Intraocular Lens (IOL), according to some embodiments of the invention;

FIGS. 4A, 4B and 4C are example schematic diagrams of images displayed to a user during a procedure, according to some embodiments of the invention;

FIG. 5 is a flow diagram of a method for a verification symbol relating to a validity of a conversion of locations between a first image of an eye of a patient and a second image of the eye of the patient, employed for ophthalmic surgery, according to some embodiments of the invention;

FIGS. 6A and 6B are schematic diagrams of a system for tool alignment, according to some embodiments of the invention;

FIG. 7 is a flow diagram of a method for a verification symbol relating to a validity of an effective alignment of a tool tracking unit with a medical tool employed in a medical procedure, according to some embodiments of the invention;

FIG. 8 is a flow diagram of a method for determining alignment of a tool tracking unit with a medical tool employed in a medical procedure, according to some embodiments of the invention; and

FIG. 9 is a schematic illustration of a conversion of the position of points of interest from a source image to a target image, according to some embodiments of the invention.

FIG. 10 shows a block diagram of a computing device which may be used with embodiments of the invention.

### DETAILED DESCRIPTION

In general, conversion of locations between coordinate systems relates herein to determining a location in a one coordinate system (e.g., a second coordinate system), which corresponds to a location in another coordinate system (e.g., a first coordinate system), or vice versa. The coordinate systems may be coordinate systems of images, tracking coordinate systems, coordinate systems of three dimensional (3D) models of body regions of interest, or coordinate systems associated with image datasets (e.g., a 3D dataset).

In general, the conversion of locations may be between two two-dimensional (2D) coordinate systems, between a 2D coordinate system and a 3D coordinate system, and/or between two 3D coordinate systems. In some embodiments, when there are two images of the same scene, the conversion of locations between coordinate systems may be conversion of locations between the two images, and specifically, determining a location in a second coordinate system being associated with a second image which corresponds to a location in a first coordinate system being associated with a first image, or vice versa, such that the spatial relationship between each of the two locations and image information in the vicinity thereof may be preserved.

In general, guidance information may be displayed. Guidance information may be, for example, displayed on an image during surgery. The guidance information may be overlaid on an image during surgery. The guidance information may include one or more of, but not limited to: models of bodily organs, hard tissue (e.g., bones) and/or soft tissue (e.g., blood vessels, nerves, tumor), models of medical tools, models of medical implants, planned positioning of a medical tool relative to a patient's body, planned trajectory of a medical tool and/or planned incision. The placement of the guidance information may be determined based on conversion of locations. The guidance information may be defined in a coordinate system of a first image and overlaid on a second image employing a conversion of locations (e.g., when the two coordinate systems are 2D coordinate systems of 2D images). In some embodiments, when both the first and the second images are 2D, the conversion of locations may be based on image registration. In other embodiments, when both the first and the second images are 2D, the conversion of locations may be carried out without image registration.

In general, conversion of locations may be prone to errors or may become invalid. In some embodiments, the invention may allow for verifying the validity of a position of overlaid guidance information, where the overlay of the guidance information is based on conversion of locations between coordinate systems. The same conversion of locations employed to overlay the guidance information may also be employed to generate a visible symbol (also referred to herein as “verification symbol”) that is also overlaid (e.g., superimposed) with the second image. The location of the verification symbol in the second image may be indicative of the validity of the conversion of locations, and thus indicative of the accuracy of the location of the guidance information on the second image, as further described herein below.

In various embodiments, the guidance information is not an overlay on the second image but may instead be vocal instructions or audio recording that is played to guide a user performing a surgical procedure. In these embodiments, a verification symbol may nevertheless be provided superimposed on the second image as long as the same conversion of locations is employed both for the program that ultimately produces the voice or audio recording for the guidance and information and for the verification symbol.

In some embodiments, the image is a two-dimensional (2D) image (e.g., a preoperative 2D image, an intraoperative 2D image, an image in a stereoscopic image pair). In some embodiments, the image is a three-dimensional (3D) image. In some embodiments, a video is a sequence of 2D images. In various embodiments, a region of interest may be viewed by a user during a procedure, this may be referred to as occurring in real-time and/or being live.

Generally, an image of a medical procedure may be displayed and/or viewed (e.g., an intraoperative image). The intraoperative image may be one or more snapshot images of the region of interest acquired during the procedure, a video of the procedure (e.g., in real time and/or live), or any combination thereof (e.g., a digital image acquired by a camera, an imaging device, and/or sensor). The intraoperative image may be an image formed optically by a viewing device (e.g., an optical image formed by a microscope, which is viewed via the microscope ocular or oculars, as further explained below).

As is apparent to one of ordinary skill in the art, an imaging device may acquire an image. The image may be streamed to a display and/or saved to memory. In some embodiments, guidance information and/or verification symbols) may be determined based on a previous frame (e.g., N-1, N-2, or N-M, where M is an integer) and superimposed (e.g., overlaid) on a current frame N.

An example procedure in which a verification symbol may be used to indicate the validity of the conversion of locations, may be an ophthalmic surgery for placement of a toric Intraocular Lens (IOL). In this example, a line indicating a preplanned orientation of the toric IOL may be provided in a coordinate system of a preoperative image of the eye (e.g., a first coordinate system associated with a first image). During the procedure, guidance information in the form of a line that corresponds to the line in the preoperative image may be superimposed on an intraoperative image in a coordinate system of an intraoperative image of the eye (e.g., a second coordinate system associated with a second image). During the procedure, the surgeon may rotate the IOL until the IOL axis marks are aligned with the preplanned orientation, as designated by the superimposed guidance information that is the line. A conversion of locations between the coordinate system associated with the preoperative image, and the coordinate system associated with the intraoperative image, may be employed to, for example, determine the location of the line in the intraoperative image (e.g., by determining the locations of the two edges of the line in the intraoperative image).

At least one image element in the preoperative image may be selected (e.g., manually by the user or automatically by a computer algorithm). The at least one image element in the preoperative image corresponds to a physical element which is assumed to be visible to the user in the intraoperative image A location in the intraoperative image corresponding to the location of the image element in the preoperative image is determined, and a verification symbol is then superimposed with the intraoperative image based on the determined location.

The at least one image element may be selected based on the type of medical procedure. For example, if a surgeon is performing eye surgery, it may be desirable to pick an image element that is in the periphery of the surgical field (e.g., blood vessel in the sclera), and not an image element that is within the limbus, as that will likely disturb the surgeon. An image element may be, for example, a prominent blood vessel, a prominent iris element and/or any other element that is within the image.

The location of an image element in the coordinate system associated with the intraoperative image may be determined by employing the same conversion of locations between coordinate systems which is employed to overlay the guidance information on the location of the image element selected in the preoperative image. Thereafter, a verification symbol may be generated and superimposed on the intraoperative image based at least on the determined location in the coordinate system associated with the intraoperative image. The verification symbol superimposed with the intraoperative image at the determined location may be an indicator as to the validity of the conversion of locations. If the verification symbol is aligned with the physical element (e.g., aligned with the image element corresponding to the physical element in the intraoperative image), then this may be an indication that the conversion of locations was accurate (e.g., valid).

In general, a plurality of image elements may be selected in the first image, and a corresponding plurality of verification symbols may be located in the second image. When the conversion of locations between coordinate systems is valid, the image element and the verification symbol may appear visually in alignment. When the conversion of locations between the coordinate systems is invalid, the image element and the verification symbol may appear visually out of alignment.

As such, a verification symbol appearing in visual alignment with the image element may provide a user with an indication relating to the validity of the conversion of the location of the guidance information (e.g., the validity of the conversion of the locations of the two edges of the line described above) from the preoperative image to the intraoperative image. As further explained below, conversion of locations between two images may be determined, for example, by an image registration process, or by a triangulation process employing common anchor points. As further exemplified below, conversions of locations may be applicable between a first coordinate system and a second coordinate system and/or between the second coordinate system and the first coordinate system.

The physical element corresponding to the selected image element employed for conversion of locations verification, may be located at a location different from the location being operated on, but within the Field of View (FOV) of the user. In some embodiments, only a symbol is overlaid at the corresponding location, and such a symbol may have a generally limited effect on the underlying image viewed by the user. The user may choose to divert their eyes to verify the validity of the conversion of locations, and such a visual verification may not interfere with the operation. The symbol may be displayed such that it is distinguishable relative to the background. This may be achieved, for example, by color (e.g., a green symbol over the white and red of the eye, during eye surgery), by shape (e.g., geometric shape not generally found in nature such as an arrow) and/or by contrast or by variable intensity (e.g., the symbol flashes or fades in and out of view). Having a distinguishable verification symbol may allow the user to verify the validity of the conversion of locations with a single glance. In some embodiments, the symbol (or symbols) may be an overlay that is limited to a small region in the surgical field (e.g., as opposed to large overlays that cover a large portion of the surgical field) so as not to obstruct the surgical field.

The image element may be manually selected by a user or automatically selected by an algorithm. For example, a neural network may be trained to select the image element (or elements). A particular algorithm may be used for selecting the image element(s) based on a type of the procedure and/or a different stage of a procedure. The different algorithms may optimize the selection, such that the verification is quick and comfortable and does not occlude the attended area, regardless of the procedure type. For example, a neural network may be trained to select segments of prominent scleral blood vessels as image elements for verification of the conversion of locations during cataract procedures. As another example, an algorithm may be configured to select segments of prominent retinal blood vessels in the periphery of the surgical field as image elements for verification during internal limiting membrane peeling procedures in vitreoretinal surgery.

As a further example, sulci (e.g., grooves in the cerebral cortex) and/or superficial blood vessels (e.g., on the surface of the cerebral cortex) may be automatically selected during open brain surgery. In this example, the elements may be selected based upon their distance from a tooltip (or tooltips), such that they do not obstruct the attended area, and the selection may be updated when the attended area changes. In these examples, when the image element is selected from a preoperative dataset, the area of exposed brain in the intraoperative image may be automatically identified (e.g., by an algorithm), and the corresponding area in preoperative dataset may be determined (e.g., based on the conversion of locations), so as to limit the area from which the image element is selected.

The selection may depend on different parameters according to user selection or system configuration. Such parameters may be the minimal and/or maximal distance between the selected element and the attended area, the type of physical elements (e.g., blood vessels or sulci), the size of the elements (e.g., the length of the blood vessel segment), the number of selected elements. The user may set the preferred visualization for the verification symbols (e.g., the type or color of the symbol and/or the transparency of the symbol), choose to enable or disable the verification overlay, and choose to enable automatic verification, as further described below. In this example, a rendered image of a model of a tumor located under the surface of the cortex may be overlaid as guidance information on the view of the surgical field (e.g., as seen via an optical surgical microscope, a digital surgical microscope and/or an exoscope), and the verification symbols may allow the surgeon to verify that the guidance information is overlaid at an accurate location.

Another example procedure in which a verification symbol may be used to indicate the validity of the conversion of locations, may be posterior segment ophthalmic surgery. In this example, a line provided with respect to the coordinate system of a preoperative image of the retina, representing a location associated with an Optical Coherence Tomography (OCT) B-scan of the retina, is superimposed on an intraoperative image of the retina during a surgical eye procedure. To that end, a conversion of locations between a first coordinate system associated with the preoperative image, and a second coordinate system associated with the intraoperative image may be determined. A verification symbol relating to the validity of the conversion of locations between the first coordinate system and the second coordinate system may be provided as described above.

As mentioned above, the conversion of locations may be prone to errors or may become invalid, regardless of the application in which it is employed. For example, when presenting information derived from a preoperative image of the eye on an intraoperative image of the eye, image registration, also referred to as image alignment, may be used to determine a conversion between the coordinate systems of the two images. Image alignment may not be sufficiently accurate when the representation of the patient region of interest in the intraoperative image differs from the representation of the patient region of interest in the preoperative image. Differences between the two images may occur, for instance, due to differences between the imaging system that generated the preoperative image and the imaging system that generates the intraoperative image, due to changes in the region of interest (e.g., changes caused by the surgical procedure), and/or due to different relative angles from which the two imaging systems acquire the images. In some cases, image alignment may be rendered unreliable (e.g., due to insufficient accuracy) due to, for example, the same differences described above.

In general, a first stage of image alignment may be finding pairs of image features, each pair consisting of one image feature in each image having a well-defined location in the image, such that the two image features in the two images may be assumed to represent the same point in the patient site of interest. For example, an image feature may be an area of pixels (e.g., 64×64 pixels), and its location may be well-defined, e.g., its location may be defined by a single point. A second stage of image alignment may be searching for a mathematical conversion that best matches locations of features in the first image with corresponding (paired) locations of features in the second image. For example, registration of images may involve finding a mathematical transformation, f(x, y)→(x′, y′), where (x, y) relates to a location in the first image and (x′, y′) relates to a location in the second image, where x, y, x′ and y′ are in units of pixels and may have integer or non-integer values. For example, if an image size is 1920×1080, x may be any number between 0 and 1920, and y may be any number between 0 and 1080.

In various embodiments, alignment is possible only locally, for instance due to distortion of the appearance of a region of interest (e.g., when gel is applied on the eye during ophthalmic surgery), or due to distortion of the region of interest itself (e.g., when pressure is applied by a tool on the region of interest). In embodiments where alignment is possible only locally, a best-fit algorithm may favor pairs of image features in one area of the image over pairs of image features in other areas. For example, the best-fit algorithm may favor pairs of image features in an area that is not distorted.

Image alignment may be based on finding a single, “global”, conversion that applies for an entire image, but in some embodiments, there may not be a single conversion that works for all the regions in the image. Since consecutive frames of the intraoperative image may slightly differ (e.g., because the patient's eye may move or a tool may move), the best fit may occasionally lock on pairs of image features from different parts of the image, which may cause jitter. In general, any method used for the conversion between coordinate systems (e.g., image alignment or other methods) may have both inherent weaknesses (as described above for image alignment) and software or algorithm errors. As such, a verification symbol may provide an indicator of the validity of the conversion of locations between coordinate systems.

Another scenario which may require symbols for verifying the validity of a conversion of locations includes medical procedures that use tracked medical tools. In some cases, the tool or tools are pre-fitted with a tool tracking unit, which enables tracking the position and orientation (P&O) of the tool in a reference coordinate system (e.g., a reference coordinate system defined by a tracking reference unit or by another object). The spatial relationship between the tool tracking unit (also referred to as “tool tracker”) and the tool is typically known.

In some embodiments, tools are not pre-fitted with tool tracking units. In these embodiments, tool tracking units are attached to the tools to provide tracking capabilities to these tools. In these embodiments, the spatial relationship between the tool tracking unit and the tool is unknown (e.g., to a required or a desired degree of accuracy) and may be determined, for example, using a calibration jig. The calibration jig may have a jig tracking unit, to allow tracking thereof. The system may employ the same tracking method to track all of the different tracking units described herein (e.g., jig tracking unit, patient tracking unit, pointer tracking unit and/or tool tracking unit) that are used for tracking a jig, patient, pointer, and/or medical tools.

A tracking unit may be components which together enable the tracking of a certain object (e.g., patient, jig, pointer, camera, tool). For example, in a system that employs reflective spheres, the tracking unit may be an array of reflective spheres, e.g., an assembly of a plurality of reflective spheres which can be coupled to the object as a single unit. For example, the tracking unit may be a plurality of individual reflective spheres which can be coupled to the object, each separately, at predetermined locations. In an example, in a system that employs in-out/out-in optical tracking, the tracking unit may include a sensor and at least one LED, as shown, for example, in U.S. Pat. No. 9,618,621 entitled “Compact Optical Tracker Having At Least One Visual Indicator Coupled to Each of Optical Tracker Sensors”, to Barak et al, which is incorporated herein by reference in its entirety. In general, the various tracking units (e.g., tool tracking unit, jig tracking unit, pointer tracking unit, patient tracking unit, and/or camera tracking unit) may be tracking units as described below.

In some embodiments, the spatial relationship between the tool tracking unit and the tool may be determined by placing the tool in the calibration jig, which is tracked, where the tool is positioned at a predetermined position and/or orientation relative to the calibration jig (e.g., the position and/or orientation of the tool in the calibration jig coordinate system is known). For example, when a distal part of a tool is elongated and straight, a tip of the tool may be placed in a divot in a calibration jig at an arbitrary orientation, allowing a determination of the tool tip location relative to the tool tracking unit. The P&O of the tool tracking unit and of the calibration jig in the tracking coordinate system may be determined by the tracking system (e.g., the P&O of the calibration jig is determined by tracking the jig tracking unit and based on the known spatial relationship between the jig tracking unit and the jig). The spatial relationship between the tool and the tool tracking unit may be calculated based on the P&O of the tool tracking unit and of the calibration jig, and the position of the tool relative to the jig. In these embodiments, the calibration jig may be pre-calibrated, e.g., the spatial relationship between a jig tracking unit and the jig may already be known.

Another technique may be used when the distal part of the tool does not exhibit rotational symmetry. In these embodiments, the tool itself may comprise a divot (or divots), and a tracked pointer (e.g., a pointer having a pointer tracking unit) may be used instead of a calibration jig. The tip of the pointer may be placed in the divot(s) of the tool, and the spatial relationship between the tool tracking unit and the tool may be calculated based on the P&O of the tool tracking unit and of the pointer, and the position of the pointer relative to the tool. In these embodiments, the pointer may be pre-calibrated, e.g., the spatial relationship between the pointer tracking unit and the pointer may already be known.

In some embodiments, a spatial relationship between a tool tracking unit and a tool may be determined by positioning the tool at various P&Os in a FOV of a camera system, as discussed in further detail below.

Determining a relative P&O between a tool tracking unit and a tool to which the tool tracking unit is attached (e.g., calculating the spatial relationship between the tool tracking unit and the tool), may be referred to as ‘tool alignment’. All (or any combination of) the above techniques for tool alignment may also be used to verify a known (or assumed) tool alignment. For example, the tool tracker may be attached to the tool using a dedicated adapter that is designed to guarantee a repeatable (e.g., known) spatial relationship between the tool tracker and the tool (e.g., a known tool alignment or an assumed tool alignment). Nevertheless, before using the tool in an image-guided (e.g., navigated) procedure, the surgeon may be required to verify that the assumed tool alignment is valid. When the above techniques are used for alignment verification, the system may compare, for instance, a divot location derived from tracking the calibration jig to the divot location derived from tracking the tool tracker unit and further based on the assumed tool alignment and the known tool shape and dimensions.

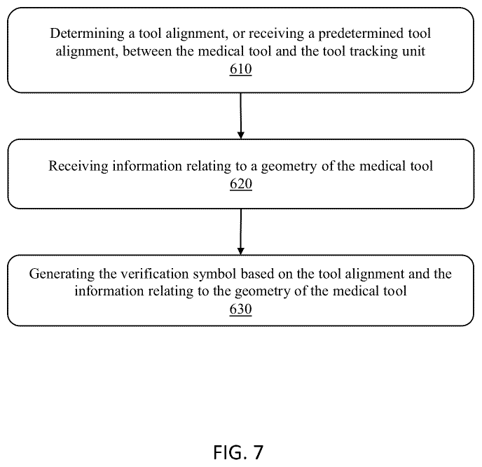

A verification symbol for a tool (e.g., a tool symbol) relating to the validity of an effective tool alignment may be provided to the user. When the tool and the tool symbol appear visually in alignment, then the effective tool alignment is valid. When the tool and the tool symbol appear visually out of alignment, then the effective tool alignment is invalid. The verification symbol relating to the validity of the effective tool alignment is described in further detail herein below (see, for example, the description of FIG. 7). Verifying the validity if the effective tool alignment using a verification symbol may be easier than using the above techniques, as it does not require pausing the surgical workflow. The tool alignment verification symbol may be overlaid on the view of the surgical field (e.g., including the tool) as seen via an optical or digital surgical microscope (e.g., an exoscope). The tool alignment verification symbol may also be overlaid on the view of the surgical field as seen via an augmented reality head-mounted display (HMD), e.g., an optical see-through HMD or a video see-through HMD.

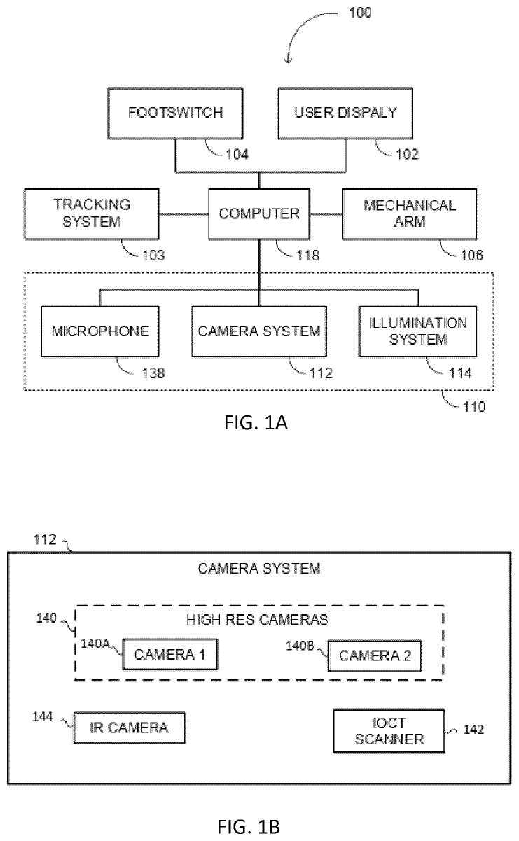

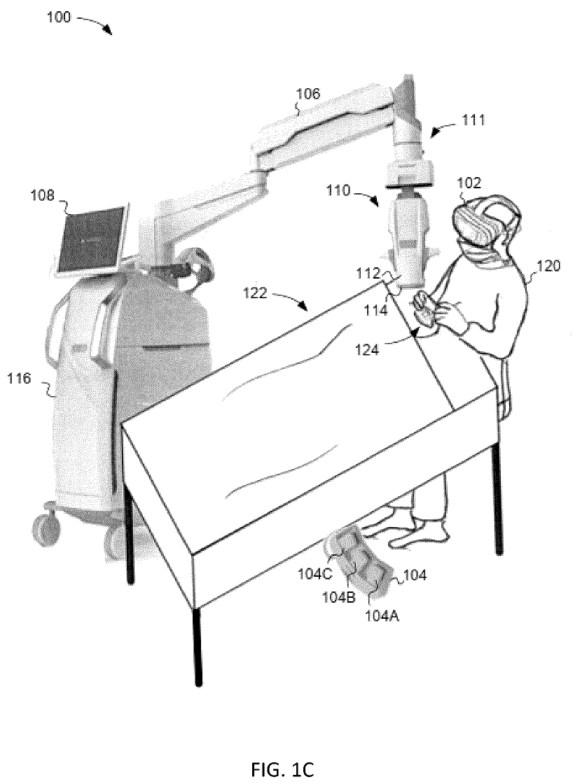

FIG. 1A is schematic block diagram of system 100, according to some embodiments of the invention. FIG. 1B is a block diagram of a camera system of FIG. 1A, according to some embodiments of the invention. FIG. 1C is a schematic illustration of an operating scenario of system 100 of FIG. 1A, according to some embodiments of the invention.

System 100 may include a user display 102. In some embodiments, as shown, for example, in FIG. 1C, the user display 102 may be an HMD. In various embodiments, the user display 102 is one or more of a wall-mounted monitor, a 2D monitor, a 3D monitor, a 3D monitor that may be viewed with special 3D glasses, a touchscreen on a cart, an HMD, and/or any display as is known in the art. As is apparent to one of ordinary skill in the art, the system of FIG. 1A-1C is a digital microscope (e.g., an exoscope), but in some embodiments, the system may be an optical surgical microscope (e.g., a standard microscope with eyepieces), or a visor-guided surgery (VGS) system (e.g., an augmented reality guidance/navigation system). In some embodiments, the system may include using a combination of one or more user displays of exoscope system as described in FIG. 1A-1C, and an optical surgical microscope and/or VGS system.

The system may include a footswitch 104. In some embodiments, the footswitch 104 may be any footswitch as are known in the art.

The system may include a cart 116 housing a computer 118 (not shown in FIG. 1C) and supporting a screen 108 (e.g., a touch-based screen). In some embodiments, where an HMD is not employed, the user display 102 and the screen 108 may be one and the same (e.g., screen 108 may serve as the user display). In some embodiments, where only an HMD is employed, the system may include only user display 102 (e.g., the system does not include screen 108).

The system may include a camera assembly 110. The camera assembly 110 may include a camera system 112, an illumination system 114 and, optionally, a microphone 138.

The system may include a mechanical arm 106. The mechanical arm 106 may include a camera assembly positioner 111). The mechanical arm 106 may be any mechanical structure that enables movement in the x, y directions.

In some embodiments, the system does not include camera assembly 110, mechanical arm 106 and camera assembly positioner 111.

In some embodiments, one or more of camera system 112, illumination system 114 and microphone 138 may be integrated in an HMD serving as user display 102. In these embodiments, the system may include also camera assembly 110, mechanical arm 106 and camera assembly positioner 111, such that some system components (e.g., camera system 112, illumination system 114, and/or microphone 138) may be included in both an HMD and a camera assembly.

In some embodiments, computer 118 is coupled to one or more of the camera assembly 110, tracking system 103, user display 102, footswitch 104 and mechanical arm 106. Each one of user display 102, footswitch 104, tracking system 103, mechanical arm 106 and camera assembly 110 that is coupled to the computer 118 may be wired or wirelessly coupled to the computer 118. The wire or wireless coupling may be by electric or fiber optic cable (not shown), Wi-Fi, Bluetooth, Zigbee, short range, medium range, long range, and microwave RF, and/or wireless optical (e.g., laser, LIDAR or infrared).

As shown in FIG. 1B, in some embodiments, camera system 112 may include a stereoscopic imager 140, which may include two cameras, for example—camera 140A and camera 140B. Camera system 112 may include an intraoperative OCT (iOCT) scanner 142 and an IR camera 144. In various embodiments, the camera system 112 does not include the OCT scanner and/or the IR camera 144. In various embodiments, the camera system 112 may include additional cameras as is known in the art, e.g., cameras for multispectral imaging. In some embodiments, camera system 112 may include a 3D sensor, such as a TOF sensor or a structured light system.

Camera system 112 may acquire images and/or stream the acquired images to computer 118. Computer 118 may process the received images (e.g., perform color correction, sharpening, filtering and/or zooming). The computer 118 may superimpose (e.g., overlay) information onto the received and processed images (e.g., guidance information, verification symbols and/or preoperative images overlaid as picture-in-picture with an interoperative image). The computer 118 may provide the processed and/or overlaid images to the user display 102 and/or the screen 108.

In some embodiments, computer 118 may control the display of the image via the user display 102 and/or the screen 108 according to a system mode, and/or any additional settings and parameters. The settings and parameters may be magnification, sharpness, region-of-interest location, color correction and/or picture-in-picture settings. System mode in ophthalmic surgery may be anterior mode, posterior mode using non-contact wide field of view lens and/or iOCT mode. Computer 118 may employ camera system 112 for other purposes as well. For example, computer 118 may employ images acquired by IR camera 114 to detect motion in the surgical field.

In addition to the images acquired by camera system 112, an image or images may be acquired preoperatively and stored in memory, rendered in real-time by a GPU (not shown), and displayed via display 102. In various embodiments, images are streamed and/or downloaded from a remote server, such as a cloud-based server. In other embodiments, the images transmitted to user display 102 are acquired from an external device, such as an endoscope.

The system may include a tracking system 103. In some embodiments, tracking system 103 may enable tracking of one or more tools, which the user employs, in a reference coordinate system. In some embodiments, tracking system 103 may enable tracking of, but not limited to: a patient, user display 102 (e.g., HMD), camera assembly 110, calibration jig, pointer, in a reference coordinate system, as described in further detail herein. Tracking system 103 may be an optical tracking system, an electro-magnetic tracking system, an inertial tracking system and/or an ultrasonic tracking system. Tracking system 103 may include at least one reference unit and at least one tracked unit. For example, a reference unit may be rigidly attached to camera assembly 110 and a tracked unit may be rigidly attached to the tool. For instance, in an optical tracking system the reference unit may be an optical detector (e.g., an infrared camera) and the tracked unit may comprise markers (e.g., infrared LEDs, reflective spheres). As another example, a reference unit may be rigidly attached to the patient, one tracked unit may be rigidly attached to camera assembly 110, and another tracked unit may be rigidly attached to the tool, thus allowing to derive the P&O of the tool relative to the camera assembly.

Optical tracking systems may utilize an array or markers. The markers may be reflectors or LEDs (e.g., infrared). The reflectors may be reflective spheres or flat reflectors (typically reflecting infrared illumination). In some embodiments, optical tracking system utilizes ARUCO markers that are not reflective. Optical tracking systems may utilize a camera or cameras that may acquire images of the markers. In some embodiments, two cameras are used to triangulate the markers, but various systems use other methods, including using a single camera.

Electro-magnetic (EM) tracking systems may be based on a transmitter generating an EM field (field generator) and receivers that may measure an EM field (e.g., EM sensor). The transmitter may be large and fixed with respect to the room, and the receivers may be small and attached to the patient and tools. Typically, they are both implemented by coils. The transmitter may generate the EM field by currents that are actively driven in the coils. The receiver measures the EM field by measuring the induced currents. Based on the known spatial characteristics of the EM field, when a receiver measures the EM field, the position and orientation of the receiver may be determined.

Tracking system 103 may acquire information relating to the P&O of the tracked object (e.g., tool) and provide computer 118 with this information. ‘Information relating to the P&O’ may include an actual P&O of the tracked object(s) in a reference coordinate system, or information from which the P&O of the object(s) may be determined. For example, when tracking system 103 is an optical tracker, the ‘information relating to the P&O’ may be the image or images acquired by the optical detector. When tracking system 103 is an electromagnetic tracker, the information relating to the P&O of the tracked object may be current measurements from receivers of the electromagnetic tracker, the voltage measurements from receivers and/or the power measurements from receivers.

In some embodiments, tracking an object (e.g., tool) may involve repeatedly determining the P&O of the object in a reference coordinate system. With reference to tracking of a tool, the P&O of a tool may be defined, for example, by defining a tool model with respect to a tool model coordinate system and defining the P&O of the tool model, to a selected number of degrees of freedom. The tool model may be a set of points in the tool model coordinate system. The tool model coordinate system may include an origin. Determining a P&O of the tool in a reference coordinate system may include, in some embodiments, determining a position of the origin of the tool model coordinate system in the reference coordinate system, and determining the orientation of the tool model coordinate system relative to the reference coordinate system. Since every point in the tool model may be defined relative to the tool model coordinate system, once the P&O of the tool in the reference coordinate system is determined, every point of the tool model may be associated with a respective position in the reference coordinate system. The position of points of interest on the tool (e.g., tool tip) may be determined in the reference coordinate system. It is noted that the model of the tool need not be a complete model of the tool. For example, when the tool is a needle, a line may be sufficient to represent the needle. In some embodiments, determining the P&O of the tool may involve determining a number of degrees of freedom for the tool model. The number of degrees of freedom may be the number needed for the particular representation of the tool, and not necessarily six. For example, if the line representing the needle is aligned with one of the axes of the tool model coordinate system, then the rotation about that axis need not be determined since the needle exhibits rotational symmetry about that axis.

Turning to FIG. 1C, shown is a user 120 observing user display 102 (e.g., HMD) to view a video (e.g., magnified video) of a surgical field 124 while performing a surgical procedure on a patient 122, for example with the aid of various tools (e.g., tracked tools and\or tools that are not tracked). In some embodiments, camera system 112 acquires a stream of images (e.g., live video) of surgical field 124, corresponding to surgical procedure performed on patient 122. Computer 118 receives and processes the stream of images and transmits the processed images to user display 102. User 120 views the images via user display 102. According to one example, computer 118 may overlay guidance information and verification symbols on the stream of images, which aids user 120 during surgery.

The system and methods described in FIGS. 1A-1C may be employed with digital systems and/or systems including surgical microscopes. For digital systems, when a verification symbol and/or guidance information (or any other information) is superimposed on an image, the pixels of the image are modified. In some embodiments that employ a surgical microscope (e.g., standard optical microscope), the verification symbol and/or guidance information (or any other information) is superimposed (e.g., via a beam splitter) on the optical image.

A surgical microscope may include two ocular viewing channels. Guidance information may be superimposed at least on one of the two ocular viewing channels of the microscope which are used to view the surgical field. For example, a first beam splitter may be positioned in one ocular viewing channel, which directs light to a camera. The camera may acquire an image of the surgical field (e.g., an image of an eye of a patient) and provide the acquired image to a computer. This acquired intraoperative image represents the optical image viewed by the user (e.g., the optical intraoperative image). Guidance information may be provided with respect to a preoperative image of the eye. The computer may employ conversion of locations between the preoperative image (e.g., a first image) and the acquired intraoperative image (e.g., a second image) to determine the location of the guidance information in the coordinate system associated with the intraoperative image (e.g., the second coordinate system).

In addition to guidance information, image elements that correspond to physical elements in a preoperative image which are assumed to be visible to the user in the optical intraoperative image may be identified in the preoperative image. A physical element may be assumed to be visible to the user if, for example, it is visually distinguishable in the preoperative image. The computer may determine the assumed locations of these identified image elements in the coordinate system associated with the acquired intraoperative image, using the same conversion of locations employed to convert the location of the guidance information. The computer may generate an overlay image comprising the guidance information and verification symbols based on the determined locations thereof in the coordinate system associated with the intraoperative image. A display may project the overlay image towards the other ocular viewing channel, where another beam splitter projects the overlay image toward the eye of the user (e.g., the user viewing the optical intraoperative image via the ocular viewing channel), thus combining the overlay image and the optical intraoperative image of the eye (e.g., overlaying the guidance information and the verification symbols on the optical intraoperative image).

The above-described process for superimposing guidance information and verification symbols in a surgical microscope may include two stages. In the first stage, the computer may use conversion of locations to convert the locations of guidance information and the image elements from the coordinate system of the preoperative image to the coordinate system of the intraoperative image (e.g., the image acquired by the camera). In the second stage, the computer may determine the location of the guidance information and verification symbols in the coordinate system of the overlay image. Regarding the second stage, it is noted that when determining the location of the guidance information and the verification symbols in the coordinate system of the overlay image, the computer may account for distortions in the optical channel of the camera (e.g., the camera that acquires the intraoperative image) and for distortions in the optical channel of the display (e.g., the display that projects the overlay image), as well as for the alignment between these two channels and the ocular viewing channel through which the overlay image is projected toward the eye of the user.

In general, a viewing device such as a surgical microscope may include an imager (e.g., camera) on either one or both optical viewing channels, and a display on either one or both optical paths, with a corresponding beam splitter or beam splitters arrangement.

The system and methods described in FIGS. 1A-1C may be employed with an optical see-through HMD or with a video see-through HMD. A surgeon donning an optical see-through HMD may have a direct (e.g., optical) view of the surgical field, including a tracked tool, augmented by guidance information that is displayed via the HMD. A surgeon donning a video see-through HMD may view the surgical field via live video from a camera, or preferably two cameras, embedded in the HMD and looking forward, the video being augmented by guidance information and displayed via the HMD. In both cases (e.g., optical or video see-through HMD), a camera or cameras embedded in the HMD may be used, for example, for tool alignment and/or tool alignment verification as further described herein below. In these embodiments, the camera system is embedded in the HMD.

As previously described, an example procedure in which a verification symbol may be used to indicate the validity of a conversion of locations, may be an ophthalmic surgery for placement of a toric Intraocular Lens (IOL). Toric IOL alignment may refer to correctly aligning a toric IOL in the lens capsule during cataract surgery. When correctly oriented, a toric IOL is typically designed to compensate for astigmatism. The IOL typically includes axis marks that indicate the IOL optical axis (e.g., a steep axis or a flat axis). According to known in the art techniques, a preoperative image is acquired by a preoperative diagnostic system. Such a preoperative diagnostic system may determine a recommended orientation of the toric IOL. The orientation may be provided in the coordinate system of the preoperative image (e.g., a desired orientation of a toric IOL may be provided as an orientation relative to the image axes). According to known in the art techniques, guidance information such as a symbol or symbols (e.g., a line or a cross) which represent the pre-planned orientation and/or the pre-planned location in which an IOL is to be positioned are superimposed on the intraoperative image. For example, a multifocal IOL may be required to be centered at a pre-planned location (e.g., along the visual axis). This pre-planned location may be determined by a diagnostic device, which provided the pre-planned location in a coordinate system of a preoperative image acquired by the device. In this example, the user moves the IOL until it is centered on a symbol representing the pre-planned location. In the example of a toric IOL, the user rotates the IOL until the axis marks located on the toric IOL are aligned with the guidance symbols indicating the pre-planned orientation. A visual representation of a planned location and orientation may be required, for example, when placing a multifocal toric IOL. According to these techniques, an IOL pre-planned orientation and/or location is provided with respect to a preoperative image or in a preoperative image coordinate system (e.g., automatically determined by a preoperative diagnostic system, or by the user). The pre-planned orientation and/or location of the IOL are then converted from the coordinate system associated with the preoperative image to the coordinate system associated with an intraoperative image. The guidance symbol may be superimposed on the intraoperative image, providing the user with a visual representation of a planned orientation and/or a planned location of the IOL.

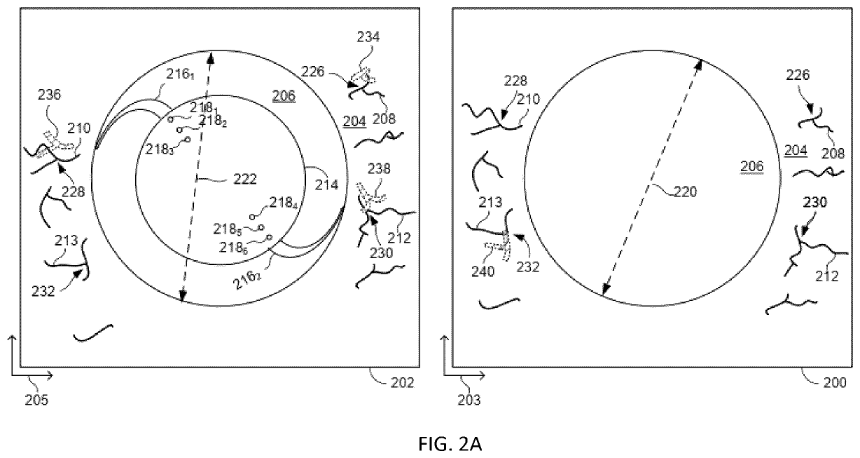

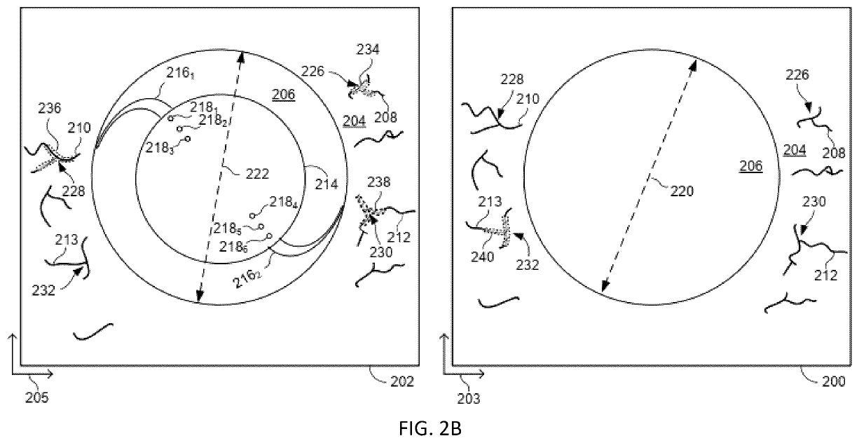

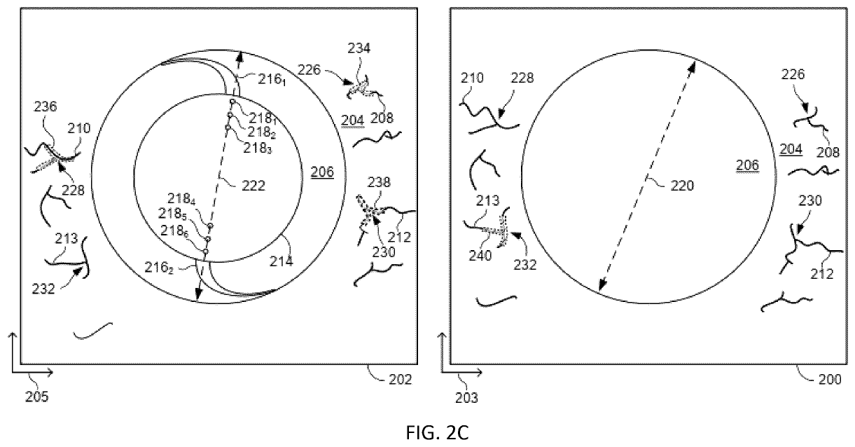

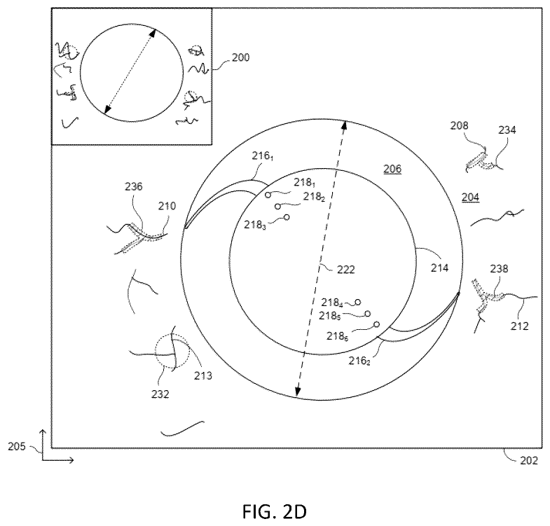

FIGS. 2A-2D are schematic diagrams of a preoperative image and an intraoperative image of an eye, during placement of a toric Intraocular Lens (IOL) 214 (e.g., using the system of FIGS. 1A-1C), according to some embodiments of the invention.

FIG. 2A represents the toric IOL placement at time TO, according to some embodiments of the invention. FIG. 2B represents the toric IOL placement at time T1 later than TO, according to some embodiments of the invention. FIG. 2C represents the toric IOL placement at time T2 later than T1, according to some embodiments of the invention. In FIGS. 2A, 2B and 2C solid lines represent image elements that were included in an originally acquired image (e.g., preoperative or interoperative images) and dashed lines represent objects (e.g., guidance information or verification symbols) that are added to the originally acquired images (e.g., via superimposition).

FIG. 2A shows a preoperative image 200 and intraoperative image 202. The preoperative image 200 is associated with coordinate system 203. The intraoperative image 202 is associated with coordinate system 205.

Presented in preoperative image 200 and in intraoperative image 202 are the sclera 204, the pupil 206 and various blood vessels (e.g., scleral blood vessels) such as blood vessels 208, 210, 212 and 213. For simplicity, the iris is not presented in the images. Preoperative image 200 may be acquired by a preoperative diagnostic system and intraoperative image 202 is acquired by a system, for example, by camera system 112 as described above in FIGS. 1A-1C.

The intraoperative image 202 also includes a toric IOL 214. The toric IOL 214 includes two haptics 2161 and 2162 intended to hold toric IOL 214 in place, once toric IOL 214 is correctly positioned. The toric IOL 214 includes axis marks 2181, 2182, 2183, 2184, 2185 and 2186. Although six axis marks are depicted in FIGS. 2A, 2B and 2C, toric IOL 214 may include any number of axis marks (e.g., two or four axis marks) allowing a user to identify the toric IOL axis. The axis marks 2181, 2182, 2183, 2184, 2185 and 2186 may also exhibit other geometrical shapes (e.g., lines and/or squares).

As described above, a preoperative diagnostic imaging system may acquire preoperative image 200. The preoperative diagnostic system may further provide guidance information, such as information relating to a recommended orientation and/or location of an IOL. The guidance information is provided, for example, as locations and/or orientations in the coordinate system of the preoperative image 200. In some embodiments, a user may receive a plurality of preoperative images and corresponding guidance information options from the preoperative diagnostic system, or from a plurality of preoperative diagnostic systems, and use these preoperative images to generate a single option. For example, different recommended orientations for a toric IOL may be employed to determine a single recommended orientation of the toric IOL (e.g., manually by the user or automatically by an algorithm). According to some embodiments, the user provides guidance information (e.g., regarding the planned orientation and/or location of toric IOL 214), for example, by providing, via a user interface, the guidance information in the coordinate system associated with the preoperative image. According to some embodiments, the user marks the guidance information on preoperative image 200 (e.g., an IOL orientation and/or location marker 220, representing the planned orientation and/or location of toric IOL 214 in the capsule). According to some embodiments, the diagnostic imaging system may automatically mark the guidance information on preoperative image 200.

During the procedure, a computer (e.g., computer 118 as described above with respect to FIGS. 1A, 1B and 1C) employs a conversion of locations between coordinate systems to convert the location of the guidance information from the coordinate system of preoperative image 200 to the coordinate system of intraoperative image 202. For example, when the guidance information is represented by a line such as line 220, the computer may copy (e.g., convert the location of) the two edges of the line from image 200 to image 202. The computer may generate a guidance symbol 222 on intraoperative image 202 according to the conversion of locations. This may present the user with an indication relating to the planned orientation and/or location of IOL 214 (e.g., as indicated by guidance symbol 222).

As mentioned above, the conversion of locations between coordinate systems may be prone to errors or may become invalid. Therefore, it may be desired to present the user with an indication relating to the validity of the conversion between coordinate system 203 and coordinate system 205. In the example brought forth in FIGS. 2A, 2B and 2C, in addition to presenting guidance symbol 222, the computer may generate verification symbols corresponding to the validity of the conversion between coordinate system 203 and coordinate system 205 on one or both of intraoperative image 202 and preoperative image 200.

The preplanned orientation of the IOL may be provided as line 220 in image 200. The computer may employ a conversion of locations to determine the corresponding location of line 220 in image 202 (e.g., by conversion of locations of the two edges of line 220). The computer may generate guidance symbol (e.g., line) 222 on image 202 based on the determined location. Image elements 226, 228 and 230 may be identified and selected either manually by the user or automatically by the computer in image 200 (e.g., the first image), and the locations thereof in coordinate system 203 may be determined (e.g., each image element may be associated with one or more locations, as described further below).

The computer may employ a conversion of locations to determine corresponding (assumed) locations of image elements 226, 228 and 230, in coordinate system 205 of intraoperative image 202 (e.g., the second image), using the same conversion of locations employed to generate guidance symbol 222 on intraoperative image 202. The computer may generate verification symbols 234, 236 and 238 to superimpose with intraoperative image 202 based at least on the corresponding locations in coordinate system 205.

In general, image elements 226, 228 and 230 are physical elements which are visibly distinct in the intraoperative image 202 (e.g., the image which the user employs during the procedure), and which are expected to have the same location with respect to the surrounding region of interest in both coordinate systems (e.g., in ophthalmic surgery the physical elements may be, for example, scleral blood vessels or visible elements in the iris that are near the outer rim of the iris). In eye surgery, the physical elements are typically visible in both the preoperative image 200 and the intraoperative image 202. In the example brought forth in FIGS. 2A, 2B and 2C, these image elements correspond to bifurcation points in scleral blood vessels in preoperative image 200.

In some embodiments, the user identifies and selects image elements 226, 228 and 230 in preoperative image 200, for example, by marking a circle around these image elements or designating these image elements employing a user interface (e.g., by pointing with a cursor). In some embodiments, the computer identifies and selects image elements 226, 228 and 230 in preoperative image 200. According to some embodiments, the computer employs image processing techniques to identify and select image elements 226, 228 and 230. For example, the computer segments preoperative image 200, and employs primitives to identify and locate prominent image elements such as bifurcation points, blood vessel segments, or prominent elements in the iris. According to another example, the computer employs a neural network or networks (e.g., machine learning or deep learning algorithms) to identify and locate the prominent image elements. Each identified image element is represented, for example, as a single point, or as a collection of image points, or as a vertex and edge or edges of a graph. As mentioned above, the computer may employ a conversion of locations to determine corresponding locations of image elements 226, 228 and 230 in coordinate system 205, and generate verification symbols 234, 236 and 238 on intraoperative image 202.

As a further example, the user may select image element 232 in intraoperative image 202, which also corresponds to a bifurcation point in blood vessels. The computer may use an inverse of the conversion of locations employed to generate guidance symbol 222 on intraoperative image 202, to determine the corresponding (e.g., assumed) location of image element 232 in coordinate system 203 of preoperative image 200. A verification symbol 240 is then presented on preoperative image 200.

In some embodiments, when an image element, identified and located in one of the images, is represented by a single point (e.g., a point indicating the location of the bifurcation of a blood vessel), the verification symbol may include an arrow pointing to a corresponding converted location of the point (e.g., using the conversion of locations) in the second image (e.g., the assumed location of the image element in the second image). In some embodiments, the image element is segmented, and locations of selected discrete points from the segment are converted to the coordinate system of the second image. The image element may be reconstructed from these discrete points and superimposed on the second image. In general, image elements may be represented, for example, as a collection of image points, or as vertexes and edges of a graph. As another example, only dots or circles are superimposed on the corresponding location in the second image.

Verification symbols 234, 236, 238 and 240 provide a user with an indication relating to the validity of the conversion of locations between the coordinate system 203 and coordinate system 205, and thus with an indication relating to the validity of the location of guidance symbol 222 in intraoperative image 202. In some cases, verification symbols 234, 236, 238 and 240 may also provide a user with information relating to the magnitude and character of the error (e.g., should such an error exist). For example, when the error is due to local distortion of the eye (e.g., due to gel applied to the eye or a tool causing deformation of the eye), one verification symbol may be visually aligned with the corresponding image element (e.g., an image element that is distant from the distorted area) while another verification symbol may appear visually out of alignment with the corresponding image element (e.g., an image element that is near the distorted area). The user may decide whether to rely on the conversion of locations or not. For example, in FIG. 2A, verification symbols 234, 236 and 238 in intraoperative image 202 and verification symbol 240 in preoperative image 200 appear visually out of alignment with bifurcations 226, 228, 230 and 232 respectively. As such, the user is provided with a visual indication that the conversion of locations between coordinate system 203 and coordinate system 205 is invalid. Therefore, the surgeon may choose not to rely on guidance symbol 222 for guidance. Consequently, the surgeon may decide to take measures to correct the situation. For instance, the source of the problem may be liquid on the eye or a tool that is causing deformation of the eye.

In FIG. 2B verification symbols 234, 236 and 238 in intraoperative image 202 and verification symbol 240 in preoperative image 200 appear visually in alignment with bifurcations 226, 228, 230 and 232 respectively. As such, the user is provided with a visual indication that the conversion of locations between coordinate system 203 and coordinate system 205 is valid. Thereafter, the user proceeds and rotates the IOL until axis marks 2181-2186 are aligned with guidance symbol 222 as depicted in FIG. 2C. In FIGS. 2A, 2B and 2C, verification symbols 234, 236, 238 and 240 appear as exhibiting a shape similar to the respective image elements (e.g., segments of blood vessels near the bifurcation points). However, in general, according to some embodiments of the invention, the verification symbols may exhibit a geometrical shape (e.g., a circle, a square, a triangle, and ellipse). The verification symbols may also exhibit the shape of an arrow pointing toward the corresponding location in the second image, as determined by the conversion of locations (e.g., when the image elements are associated with a single location). The verification symbols may also exhibit the shape of brackets around the corresponding location in the second image. In some embodiments, one verification symbol may be associated with more than one image element (e.g., when the image elements are in close proximity to each other). In some embodiments, the image element may be a contour of an anatomical element, such as the limbus, and the verification symbol may exhibit the shape of that contour. In these embodiments, once the contour is identified in the first image it may be represented as multiple points (e.g., 10 points uniformly distributed along the contour), and these points may be converted to the coordinate system of the second image. The computer may reconstruct the contour in the coordinate system of the second image and overlay the reconstructed contour on the second image as a verification symbol. In the embodiments where the contour of an anatomical element is the contour of the limbus, the computer may determine an ellipse that best fits the corresponding locations in the coordinate system of the second image and employ this ellipse as a verification symbol.

FIGS. 2A-2C represent a possible layout of images that are displayed to the user. In this layout, the user views the preoperative image 200 and the intraoperative image 202 in a side-by-side layout. FIG. 2D depicts another possible layout, in which image 200 is presented as superimposed on image 202 in a picture-in-picture (PIP) layout. However, when a surgeon prefers to view only the intraoperative (live) image, all the image elements may be selected in the preoperative image, and corresponding verification symbols are superimposed only on the intraoperative image. Since the image elements employed for the verification of the conversion of locations between coordinate systems are distinct (e.g., clear and distinct bifurcation points, or, for example, the contour of the limbus), it may be sufficient that the surgeon views only the intraoperative image. In embodiments that the conversion of locations between coordinate systems is not valid, the surgeon may see that the verification symbol is not aligned with the respective image element. In general, in the embodiment described in conjunction with FIGS. 2A-2D, the surgeon may select their preferred presentation mode (e.g., PIP, picture by picture, intraoperative image only).

In some embodiments, the surgeon may be provided with guidance information with respect to the preoperative image. For example, in surgical navigation systems (e.g., for brain surgery) in which a tool is tracked, the guidance information may include a symbol representing the tool P&O, superimposed on images generated from preoperative CT or MRI scans. In cataract surgery, the guidance information may include guidance symbols displayed as overlay on the preoperative image instead of on the intraoperative image. For example, the preoperative image may be overlaid with two guidance symbols (or groups of symbols). The first symbol (e.g., a line) may represent the preplanned IOL orientation and/or location with respect to the preoperative image (e.g., as determined by a preoperative diagnostic device). The second symbol or group of symbols (e.g., a set of six dots) may represent the actual IOL orientation and/or location as converted from the intraoperative image (e.g., using the conversion of locations). In the case of a toric IOL, the computer detects the IOL axis marks in the intraoperative image. The locations of these IOL axis marks are converted from the intraoperative image to the preoperative image employing a conversion of locations. Axis marks designators are then overlaid on the corresponding converted locations in the preoperative image, representing the actual orientation and location of the toric IOL. The user may move and rotate the IOL until the two symbols (e.g., the line and the six dots) are aligned. In addition to the guidance symbol, the computer may also overlay the preoperative image with verification symbols that are generated based on image elements in the intraoperative image, similarly to as described with relation to FIG. 2A-2D. According to another embodiment of the invention, verification symbols, which present the user with an indication regarding to the validity of the detection of the IOL axis marks, are superimposed on the intraoperative image. These verification symbols indicate that the axis marks are correctly identified.

When the intraoperative image is stereoscopic, the verification symbol may be generated for each of the left and right stereoscopic images, such that the two 2D verification symbols appear as a single 3D verification symbol when overlaid with the stereoscopic image.

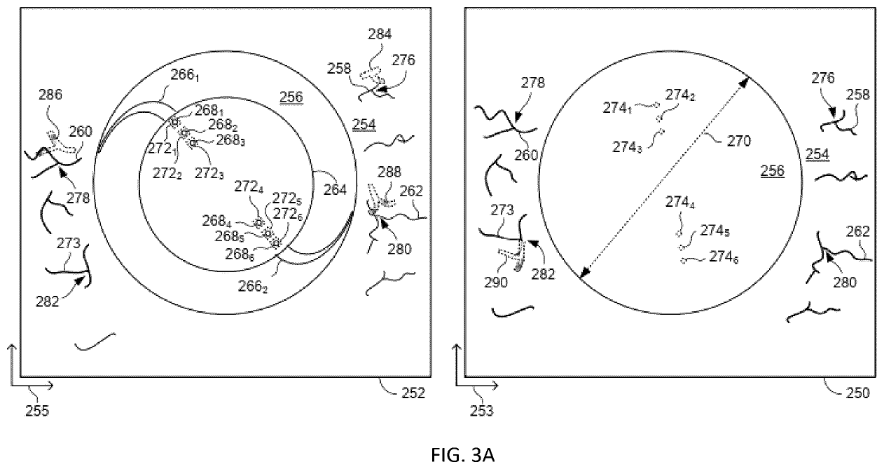

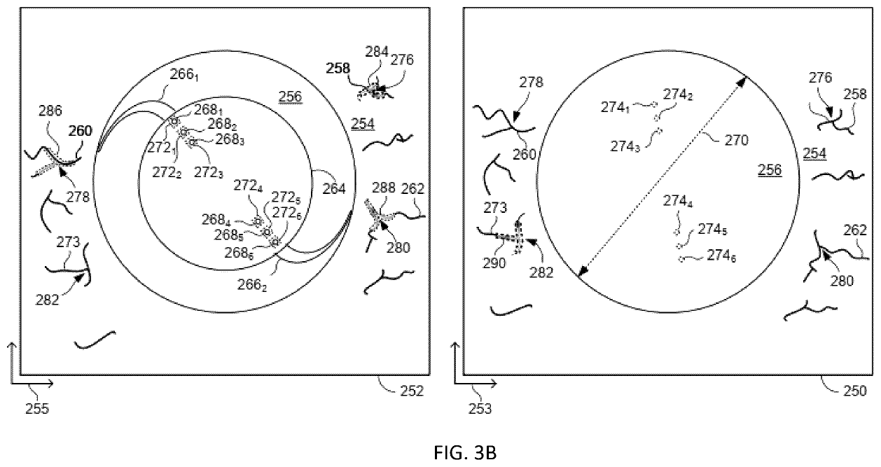

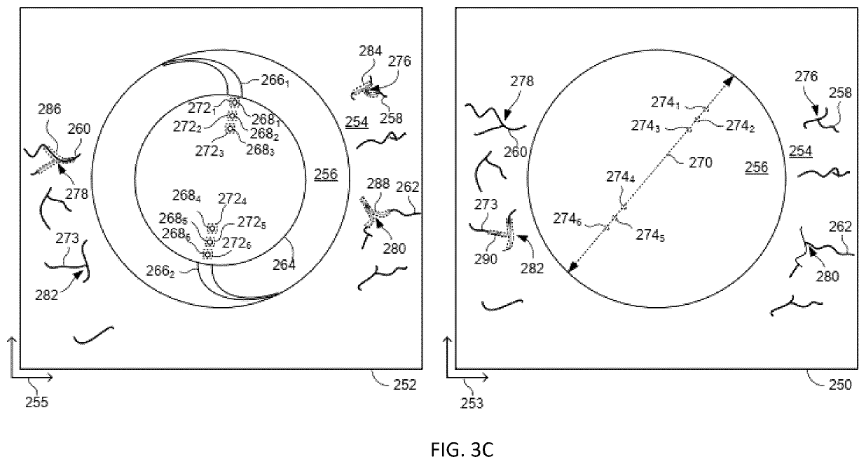

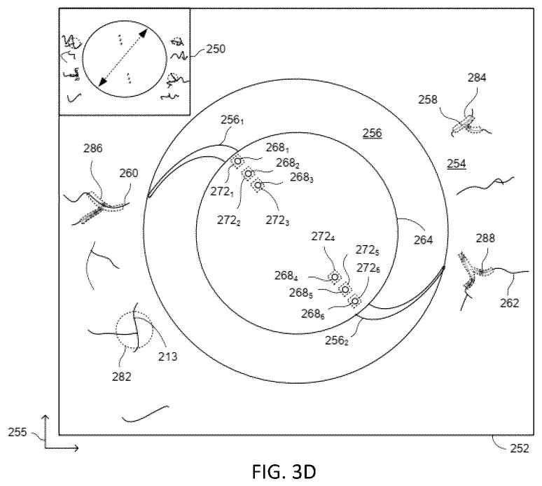

FIGS. 3A-3D are schematic diagrams of a preoperative image 250 and an intraoperative image 252 of an eye, during placement of a toric Intraocular Lens (IOL) 264, according to some embodiments of the invention.

FIG. 3A represents the toric IOL placement at time TO, FIG. 3B represents the toric IOL placement at time T1 later than TO, and FIG. 3C represents the toric IOL placement at time T2 later than T1. In FIGS. 3A-3D solid lines represent image elements that were included in original acquired intraoperative image and dashed lines represent objects that are added (e.g., overlaid) to the acquired intraoperative image.

Toric IOL 264 includes two haptics 2661 and 2662 intended to hold toric IOL 264 in place, once toric IOL 264 is correctly positioned. Toric IOL 214 includes axis marks 2681, 2682, 2683, 2684, 2685 and 2686. Although six axis marks are depicted in FIGS. 3A-3D, toric IOL 264 may include any number of axis marks (e.g., two or four axis marks) allowing a user to identify the toric IOL axis. The axis marks 2681, 2682, 2683, 2684, 2685 and 2686 may also exhibit other geometrical shapes (e.g., lines and/or squares). Presented in preoperative image 250 and in intraoperative image 252 are the sclera 254, the pupil 256 and various blood vessels (e.g., scleral blood vessels) such as blood vessels 258, 260, 262 and 273. For simplicity, the iris is not presented in the images. Preoperative image 250 is acquired, for example, by a preoperative diagnostic system and intraoperative image 252 is acquired, for example, by camera system 112 (e.g., FIG. 1A). Preoperative image 250 is associated with coordinate system 253. Intraoperative image 252 is associated with coordinate system 255.

Similar to as mentioned above, a preoperative diagnostic imaging system acquires preoperative image 250. Such a system or systems may further provide information relating to a recommended orientation and/or location of the toric IOL.

During the procedure, computer 118 identifies axis marks 2681-2686 in the intraoperative image 252 (e.g., the first image), and determines locations thereof in coordinate system 255. Computer 118 generates first axis marks designators 2721, 2722, 2723, 2724, 2725, and 2726 on the corresponding location of each of axis marks 2681-2686. This presents the user with an indication regarding a correctness of the detection of axis marks 2681-2686 in intraoperative image 252. In case axis marks designators 2721-2726 are not aligned with the corresponding location of each of axis marks 2681-2686, the surgeon (or user) knows not to trust any guidance provided based on the detected locations. The computer 118 may determine the corresponding locations of axis marks 2681-2686 in coordinate system 253 of preoperative image 250 (e.g., the second image) employing the conversion of locations between coordinate systems.

The computer 118 may generate second axis marks designators 2741, 2742, 2743, 2744, 2745 and 2746 on preoperative image 250 at least based on the corresponding locations of axis marks 2681-2686 in coordinate system 253. This may present the user with an indication relating to the actual orientation and/or location of toric IOL 264 relative to the pre-planned orientation and/or location, as indicated by marker 270.

Similar to the description above, the conversion of locations between coordinate systems may be prone to errors or may become invalid. Therefore, it may be desired to present the user with an indication relating to the validity of the conversion between coordinate system 253 and coordinate system 255. In the example of FIGS. 3A-3D, in addition to presenting second alignment designators 2741-2746, verification symbols corresponding to the validity of the conversion between coordinate system 253 and coordinate system 255 are presented. For example, image elements 276, 278 and 280 are identified and selected in preoperative image 250 (e.g., by computer 118 or by the user, for example, by marking a circle around these image elements). Image elements 276, 278 and 280 are similar to image element 226, 228 and 230 described above in conjunction with FIGS. 2A-2C and are selected in a similar manner.

The computer 118 may determine the locations of image elements 276, 278 and 280 in coordinate system 253 of preoperative image 250. The computer 118 may also determine the corresponding (assumed) locations of image elements 276, 278 and 280 in coordinate system 255 of intraoperative image 252, using the same conversion of locations employed to determine the corresponding locations of axis marks 2681-2686 in coordinate system 253. The computer 118 may generate verification symbols 284, 286 and 288 at the corresponding (assumed) locations of image elements 276, 278 and 280 in coordinate system 255 of intraoperative image 252. Similarly, image element 282 may be identified and selected in intraoperative image 252. Thereafter, computer 118 determines the location of image element 282 in coordinate system 255 of intraoperative image 252. Computer 118 also determines the corresponding (assumed) location of image element 282 in coordinate system 253 of preoperative image 250, using the same conversion of locations employed to determine the corresponding locations of axis marks 2681-2686 in coordinate system 253. A verification symbol 290 is then presented on preoperative image 250 at the corresponding (assumed) location of image element 282 in coordinate system 253.

Verification symbols 284, 286, 288 and 290 provide a user with an indication relating to the validity of the conversion between the coordinate system 253 and coordinate system 255, and thus with an indication relating to the validity of the location of axis marks designators 2741-2746. For example, in FIG. 3A, verification symbols 284, 286 and 288 in intraoperative image 252 and verification symbol 290 in preoperative image 250 appear visually out of alignment with bifurcation 226, 228, 280 and 282 respectively. For example, a nurse applied drops on the eye which distorted the image or the surgeon applied (e.g., pushed or pulled) a tool and deformed the eye. Thus, prior to aligning toric IOL 264, the user is provided with a visual indication that the conversion of locations between coordinate system 253 and coordinate system 255 is invalid and the location of axis marks designators 2741-2746 do not correspond to the location of axis marks 2681-2686. Upon identifying that the conversion between coordinate system 253 and coordinate systems 255 is invalid, the user may wait until the eye returns to former state (e.g., no drops and no tool applied) or take other corrective action or actions. Thereafter, in FIG. 3B, verification symbols 284, 286 and 288 in intraoperative image 252 and verification symbol 290 in preoperative image 250 appear visually in alignment with bifurcation 276, 278, 280 and 282 respectively. As such, the user is provided with a visual indication that the conversion between coordinate system 253 and coordinate system 255 is valid and the user may proceed with the alignment procedure. In FIG. 3C, axis marks designators 2741-2746 are aligned with marker 270. In FIGS. 3A, 3B and 3C, image 250 and 252 were presented as picture by picture. However, and with reference to FIG. 3D, image 250 may be presented with image 252 as Picture In Picture (PIP), where image 250 is presented in image 252. In general, similar to as described above in conjunction with FIGS. 2A-2D, the surgeon may select their preferred presentation mode (e.g., PIP, picture by picture, intraoperative image only) or opt to receive auditory guidance. Auditory guidance may be generated (e.g., by computer 118) for instance by calculating the angular distance between the actual IOL orientation and the preplanned IOL orientation. The auditory guidance may consist, for instance, on a sound having a frequency which changes as a function of the alignment. Similar to as described above, the preplanned orientation may be provided for instance by the diagnostic device that generated the preoperative image, and the actual orientation may be derived for instance by determining a line that best fits the six dots in the preoperative image coordinate system.

FIGS. 2A-2D and 3A-3D above are related to an example where a verification symbol relating to the validity of conversion of locations between a coordinate system associated with a preoperative image and a coordinate system associated with an intraoperative image is presented to the user. Nevertheless, a verification symbol relating to the conversion of locations between coordinate systems associated with two intraoperative images may also be displayed. For example, in case of two users (e.g., a senior surgeon and a resident surgeon), one user draws a guidance symbol (e.g., a desired location of an incision) on an intraoperative image (e.g., a snapshot of the live video). This guidance symbol is to be presented on the live video for the other user. To that end, a conversion of locations is employed to present the guidance symbol on the live video. A verification symbol relating to the validity of conversion of locations between the respective coordinate systems is also presented, similar to as described above.

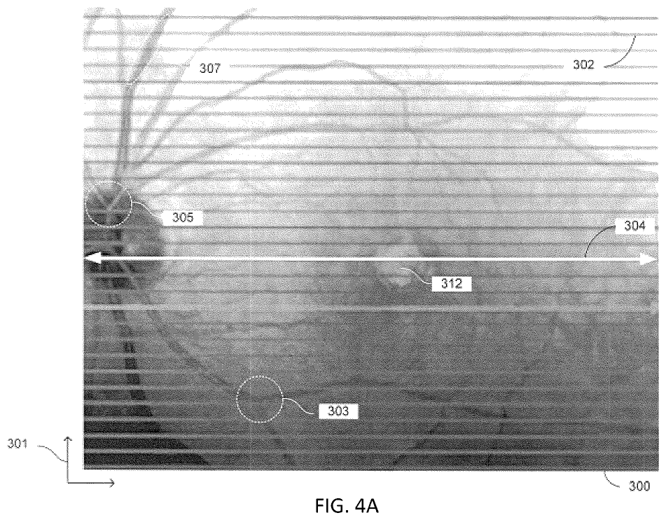

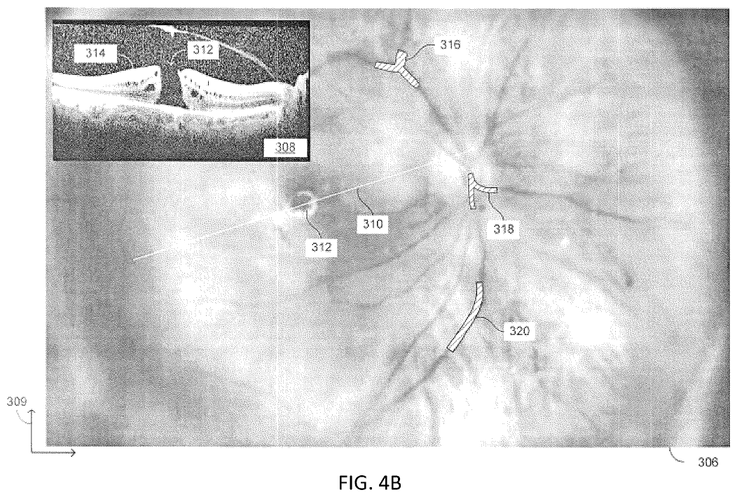

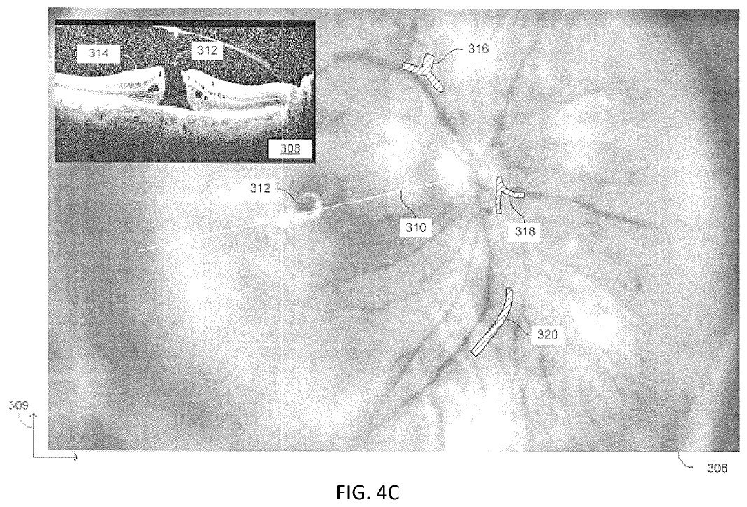

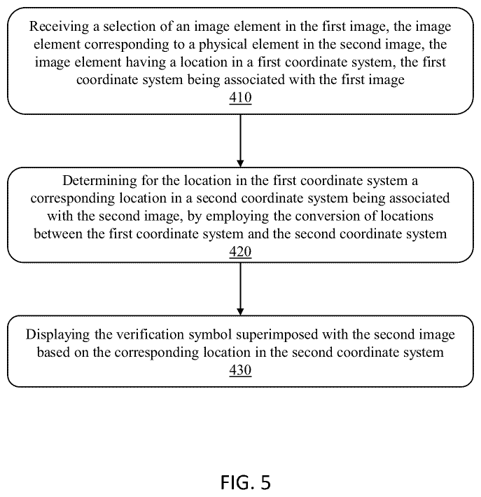

FIGS. 4A-4C are schematic diagrams of images displayed to a user during a procedure, according to some embodiments of the invention. FIG. 4A shows a pre-operative image 300 of a retina associated with a respective coordinate system 301. FIGS. 4B and 4C are schematic illustrations of images displayed to a user during a procedure, in accordance with some embodiments of the invention.

Image 306 is a live video (e.g., intraoperative image) of a retina acquired by a camera system (e.g., camera system 112 as described above with respect to FIGS. 1A, 1B and 1C) and associated with a respective coordinate system 309. Image 308 is an OCT B-scan acquired, for example, by a diagnostic (e.g., preoperative) OCT device. Image 300 is acquired by the same OCT device as used for image 308 concurrently with the acquisition of multiple B-scans, including B-scan 308.

Depicted on image 300 are lines, such as line 302, which represent the locations on the retina corresponding to the multiple B-scans acquired by the OCT device. Line 304 represents the location in image 300 corresponding to cross section (B-scan) image 308 (e.g., FIG. 4B). The OCT device may also generate overlaying lines corresponding to the various B-scans. The information relating each of the various B-scans to one of the various lines may be provided separately (e.g., via a text file).

The image of the retina may be provided without the overlaid lines, and the information regarding the location with respect to the image of the retina corresponding to each B-scan may be provided separately, for example as locations of lines in the coordinate system of the image of the retina, provided for instance as two (x, y) pixel locations representing two edges of a line for each B-scan. Depicted in images 300, 306 and 308 is a macular hole 312 in the retina of the eye.

Image 300 and image 306 are acquired in different dispositions of the cameras relative to the patient and as such, appear in FIGS. 4A and 4B to be rotated by approximately 180 degrees. Image 306 is displayed to a user with image 308 displayed in PIP. Overlaid on image 306 is a line 310, indicating the location corresponding to cross section image 308 on image 306. Line 310 and cross section image 308 provide guidance information for the surgeon. To present line 310 at the correct location and/or orientation in image 306, a conversion of locations between coordinate system 301 and coordinate system 309 may be determined. However, it may be desired to present the user with a visual indication relating to the validity of the conversion of locations between the coordinate systems. Therefore, a plurality of image elements representing physical elements, such as bifurcation points 303 and 305 and blood vessel segment 307, may be identified in image 300 (e.g., the first image), and the locations thereof in coordinate system 301 may be determined.

The same conversion of locations between coordinate systems used to determine the location of line 310 in coordinate system 309 may be employed to determine the locations of these image elements in coordinate system 309 of intraoperative image 306 (e.g., the second image).

A verification symbol respective of each one bifurcation points 303 and 305 and blood vessel 307 may be superimposed at least based on the corresponding location (or locations) thereof in coordinate system 309 (e.g., on image 306). Verification symbol 316 is presented at the location corresponding to bifurcation point 303. Verification symbol 318 is presented at the location corresponding to bifurcation point 305 and verification symbol 320 is presented at the location corresponding to blood vessel segment 307.

With reference to FIG. 4B, when verification symbols 316, 318 and 320 appear visually in alignment with the corresponding image elements thereof, the conversion of locations between coordinate systems is valid. With reference to FIG. 3C, when verification symbols 316, 318 and 320 appear visually out of alignment with the corresponding image elements thereof, the conversion of locations between coordinate systems is invalid. It is noted that to determine the conversion of locations between the preoperative image and the intraoperative image, a computer (e.g., computer 118 of FIG. 1A) employs a preoperative image of the retina without the overlaid lines. This preoperative image of the retina without the overlaid lines may also be provided by the diagnostic OCT device (e.g., identical to image 300 but without the superimposed lines). The information regarding the location corresponding to each of the B-scans, with respect to the retina, may be provided by the OCT device as coordinates in coordinate system 301.

FIGS. 2A-2D and 3A-3D as described above relate to converting locations related to overlay data (e.g., guidance information, augmentations, and/or verification symbols) that are defined with respect to a first 2D image coordinate system, to a second 2D image coordinate system. In some embodiments, it may be desired to overlay data on a 2D image that is defined with respect to 3D datasets. In various embodiments, 3D datasets may include a plurality of 2D slice images from CT, MRI, angiographic and/or ultrasound imagers (e.g., in brain and spine surgery). In various embodiments, 3D datasets may include a plurality of 2D B-scans acquired by an OCT imaging device and/or a plurality of 2D images acquired by a Scheimpflug imaging device (e.g., in ophthalmic surgery). 3D datasets also relate to a combination of datasets employing modality fusion (e.g., either a single combined dataset, or separate datasets all registered to a single ‘combined’ coordinate system), or information derived from such 3D datasets. The terms ‘3D image information’, ‘3D guidance information’ or ‘3D information’ may relate herein to the 3D dataset and to information derived from the 3D dataset. For example, a 3D model that was derived from the 3D dataset (e.g., a 3D segmentation of a tumor derived from an MRI scan), an oblique slice that was derived from the 3D dataset, a rendered 2D image of a 3D model that was derived from the 3D dataset, and preplanning information that was determined (e.g., by a surgeon or automatically by an algorithm) based on the 3D dataset (e.g., a planned trajectory, a planned incision). Preplanning information may be visually represented as zero-dimensional preplanning information (e.g., a point representing a center of a tumor), as one-dimensional (1D) preplanning information (e.g., a line representing a planned trajectory of a tool), as 2D preplanning information (e.g., a plane, a surface, an incision on a surface), or as 3D preplanning information (e.g., a volume that is to be ablated or drained). Information that is derived from a 3D dataset is defined with respect to the same 3D coordinate system of the 3D dataset.

In some embodiments, 3D image information may be superimposed on an image or images (e.g., live video) acquired during the procedure in a variety of cases. For example, during brain surgery using a microscope or an endoscope, a rendered image of a model of a tumor, derived from a 3D dataset, is superimposed at a corresponding location on an image of the region of interest. In some embodiments, during a laparoscopic procedure, an oblique slice image of an organ (e.g., the liver or the kidney) derived from a 3D dataset is superimposed on a corresponding location in a live video of the region of interest. In some embodiments, a planned trajectory of a tool is superimposed on a stereoscopic image pair (e.g., when the trajectory is planned based on 3D datasets such as CT or MRI scans). Superimposing 3D information on a corresponding location in an image (or images) of the region of interest is performed based on a conversion of locations between the coordinate system associated with the 3D dataset and the coordinate system associated with each image. Several examples of such a conversion of locations are described herein below.

In some embodiments, a verification symbol or symbols, relating to the validity of the conversion of locations, are presented to the user, providing the user with a visual indicator relating to the validity of the conversion of locations. The visual indicator may be superimposed on the image or images (e.g., an intraoperative image), or on the 3D image information (e.g., on an oblique slice that was derived from the 3D dataset, or on a rendered 2D image of a 3D model that was derived from the 3D dataset). To present such a visual indicator as an overlay on the live image (e.g., the live video from an endoscope, or an image generated by a microscope), selected elements may be identified (e.g., either by the user or automatically) in the 3D image information. These elements may be at least partially visible, or assumed to be at least partially visible, in the live image. When the live image is stereoscopic, the verification symbol may be generated for each of the left and right stereoscopic images, such that the two 2D verification symbols appear as a single 3D verification symbol when overlaid with the stereoscopic image. In some embodiments, to present such a visual indicator or indicators as an overlay on the 3D image information, selected elements are identified in the live image. These elements may be at least partially visible, or assumed to be at least partially visible, in the 3D image information.

The identified elements may be naturally occurring, for example, blood vessels, an organ or organs, surfaces or contours of organs or bones. For example, when the identified element is the surface of a bone, the corresponding verification symbol may be a wire frame surface encompassing the bone. The identified elements may also be artificial elements such as fiducial markers or implants. In general, any distinct element may be selected. Once the image elements are identified and selected in one of the live image or the 3D image information, a verification symbol is overlaid at a corresponding location in the other one of the image or the 3D information, using the same conversion of locations employed to superimpose the 3D information. These verification symbols provide the user with a visual indication relating to the validity of the conversion of locations. When the conversion of locations is valid, the verification symbol appears in visual alignment with the corresponding element. When the conversion of locations is invalid, the verification symbol appears visually out of alignment with the corresponding element.

Following is an example relating to neurosurgery. During a neurosurgical procedure, it may be desired to present the user with 3D guidance information superimposed on a live image of the brain (e.g., a 2D image during a minimally invasive endoscopic procedure, or a stereoscopic image pair during an open brain surgery using a microscope). The 3D guidance information may be, for example, a rendered image of a 3D model or a rendered image of selected elements in a 3D model, such as, for instance, a model of a tumor to be treated, and/or a model of blood vessels, which is generated from a 3D dataset. The 3D model may include hard tissue (e.g., bones), soft tissue (e.g., an organ, a tumor, blood vessels or nerves), or both. The 3D model may include anatomical elements such as the nose and ears, and/or further include fiducials, which may be employed for determining a conversion of locations, as described further below. The 3D guidance information may also be, for example, preplanning information (e.g., a trajectory of a medical tool). The 3D guidance information is associated with a respective 3D coordinate system.

To superimpose the 3D guidance information on the acquired image, a conversion of locations between the coordinate system associated with the 3D guidance information, and the coordinate system of the image is determined (e.g., as described further below). In addition to presenting the 3D guidance information (e.g., the rendered images of a tumor or a preplanned trajectory of a medical tool, such as a needle) as an overlay on the live image, it may also be desired to present the user with an indication relating to the validity of the conversion of locations. As such, an element or elements which are visible or assumed to be visible in the live image and which are identified in the 3D information (e.g., not necessarily in the 3D model), are selected. For example, during an open brain surgery, such an element or elements may be a blood vessel or vessels. According to another example, during an open brain surgery, these elements may be cortical gyri. A verification symbol of the gyri or blood vessels, identified in the 3D information, is overlaid at the corresponding (e.g., assumed) location thereof in the acquired live image, employing the same conversion of locations that was used for overlaying the 3D guidance information on the image. In some embodiments, fiducial markers which may be identified in the 3D model or the 3D information, and which are visible in the acquired image, are identified in the 3D image information, and a verification symbol corresponding thereto is overlaid at the corresponding (e.g., assumed) location thereof in the acquired image employing that same conversion. In general, the elements in the 3D model, which may be employed for guidance and for verification of the conversion of positions, may change (e.g., either automatically or by the user) during different stages of the procedure.

The conversion of locations between the 3D model and the image of the brain (e.g., between the coordinate system of the 3D image information and the coordinate system of the live image) may be achieved in several ways. In some embodiments, registering the 3D image information to a reference coordinate system associated with a tracking system, and tracking the camera (or cameras) that generate the image (or images) with the tracking system. In these embodiments, images of the 3D information (e.g., of 3D models that are part of the 3D information) may be rendered from the point-of-view (POV) of the camera (or the two POVs of the two cameras, in the case of a stereoscopic image), where the POV is the relative P&O of the camera with respect to the 3D information, that is derived from the registration and tracking data. The rendered images may additionally be processed employing known (e.g., pre-calibrated) optical characteristics of the cameras, such as the FOV and optical distortions, such that these rendered images correspond to the images acquired by the cameras.

In some embodiments, the distortions of the live image may be corrected before overlaying the 3D guidance information and the verification symbols, and before streaming them to the display. In either case, the 3D guidance information and the verification symbols may be overlaid at the corresponding locations thereof on the live image. Although the conversion of locations between the 3D model and the image of the brain described herein above may not relate to an explicit transformation f(x, y, z) (x′, y′), this method may relate to conversion of locations between coordinate systems, and locations in the coordinate system of the 3D model may be converted to locations in the coordinate system of the image.

In some embodiments, locations in the coordinate system of the live image may be converted to locations in images that are rendered from the 3D guidance information (e.g., rendered images of a 3D model that is derived from the 3D information, an oblique slice generated from the 3D information, and the like), thus allowing to overlay on the 3D information both guidance information (e.g., the location of a tracked tool) and verification symbols (e.g., the location of a blood vessel) that are defined with respect to the live image.

In brain surgery, a reference coordinate system is defined by a tracking reference unit which is in a fixed spatial relationship with the head of the patient. At the beginning of a procedure, a transformation between the coordinate system associated with the 3D information and the reference coordinate system may be determined (e.g., ‘registration’, which may be different from ‘image registration’ described earlier). When the 3D dataset or the 3D model include representations of fiducials that are still adhered to the patient, the registration may be performed by placing the tip of a tracked tool in the centers of the adhered fiducials and recording the locations thereof in the reference coordinate system.

A surgeon may identify the locations of the fiducials in the coordinate system associated with the 3D dataset. For example, the 3D information is displayed on the screen and the user employs a cursor to designate the fiducials. The computer may determine the transformation between the coordinate system of the 3D dataset or the 3D model and the reference coordinate system based on the locations of the fiducials in the two coordinate systems. In some embodiments, alternatively or in addition to using fiducials, the surgeon may point to anatomical elements that are distinct in the 3D dataset. In some embodiments, 3D mapping is relied upon for part of the patient's face to generate a 3D surface and match this surface with the corresponding surface generated from the 3D dataset.

When a stereoscopic imager is employed, such as stereoscopic imager 140 of FIG. 1B, the position and orientation of each of stereoscopic cameras 140A and 140B in the reference coordinate system may be determined. In some embodiments, the position and orientation of stereoscopic imager 140 is tracked by a tracker unit attached to the imager at a known (e.g., predetermined or calibrated) location thereon. A tracker system may track the position and orientation of the tracker unit in the reference coordinate system, relative to the tracker reference unit, and the position and orientation of the stereoscopic imager in the reference coordinate system may be determined from the position and orientation of the tracker unit. The P&O of the 3D model in the coordinate system of the stereoscopic imager may be determined, and consequently elements from the 3D model may be rendered from the POV of the camera (or cameras) to generate the guidance overlays and the verification symbols. A verification symbol or symbols corresponding to the abovementioned element or elements employed for verification (e.g., blood vessels, gyri or fiducials) may be superimposed on the stereoscopic image pair and may provide a visual indication relating to the validity of the conversion of locations.

In some embodiments, different 3D models are employed for generating the guidance information and for generating the verification symbol (e.g., both models are generated from the same 3D dataset and share a common coordinate system). For example, when generating the 3D guidance information (e.g., an overlay showing a tumor that is not visible in the image acquired by the camera), the computer may render an image of a 3D model of the tumor, generated from the 3D dataset. When generating the verification symbol, the computer may render an image of a 3D model of superficial cortical blood vessels (e.g., blood vessels located on the surface of the cortex) that are assumed to be visible at that stage of the procedure (e.g., after revealing the cortex), that was generated from the same 3D dataset as the 3D model of the tumor. In some embodiments, a single 3D model is employed for generating both the guidance information and the verification symbol. For example, the verification symbol (or symbols) are rendered from those parts of the 3D model that are assumed to exhibit a direct line of sight with the camera (e.g., a surface that is “visible” to the camera), whereas the 3D guidance information may be rendered from the parts of the 3D model that may be at least partially hidden in the image, and therefore augment the visible FOV of the user (e.g., the 3D model may include different layers and/or different segments that may be employed separately). In some embodiments, both the guidance information and the verification symbol are rendered together. In the examples above, elements within the 3D model may be selected, and only these elements may be rendered. For example, when employing a 3D model of superficial cortical blood vessels, a selected element may be a short segment of a blood vessel that is at the periphery of the surgical field (e.g., as appearing in the live image). The 3D guidance information may also be rendered from preplanning information that was added to the 3D dataset and is not part of the raw imageries.

In the example of brain surgery, brain shift may be a problem where the intraoperative position of the brain is shifted with respect to its position at the time in which the 3D dataset was captured. The shift may be relative to the skull, and specifically relative to elements in the preoperative data that were used for registration. Brain shift may occur both in open brain surgery and in minimally invasive endoscopic procedures (such as endoscopic skull base procedures), but may be especially predominant after craniotomy (e.g., after a section of the skull is removed) in open brain surgery. In such cases, the determined position and orientation of parts of the preoperative 3D model (e.g., parts thereof other than the skull) in the reference coordinate system may not correspond to the actual position and orientation of the corresponding region of interest. For example, the representation of the tumor presented to the user may not coincide with the tumor.

As described above, a visual indication relating to the validity of the position and orientation of the 3D model in the reference coordinate system may be provided, for example by employing the gyri or sulci or one or more superficial blood vessels (e.g., on the surface of the cerebral cortex). When brain shift occurs, the verification symbols do not coincide with the corresponding elements, thus providing a visual indication that the conversion of locations is invalid. The verification symbols may also provide a quantitative measure on the amount of brain shift and allow the surgeon to compensate for the brain shift while still relying on the 3D guidance information.

The conversion of locations in the example above was based on registering the 3D information to a tracker reference unit and tracking the camera. The P&O of the 3D information in the camera coordinate system may be derived, allowing rendering of images from the 3D information, such that the coordinate system of the rendered images is registered with the coordinate system of the actual images acquired by the camera. Described herein are two embodiments that include alternative methods for deriving the P&O of the 3D information in the camera coordinate system. These methods may alleviate problems that arise from brain shift in the tracker-based method. The first embodiment is suitable for both a single 2D image (e.g., generated by a standard endoscope or laparoscope) and a stereoscopic image pair (e.g., generated by a microscope or by a stereoscopic endoscope). The second method requires a stereoscopic image pair or alternatively 3D image information generated by a 3D sensor such as a TOF sensor or a structured light system. Both methods may rely on iteratively improving an estimated P&O of the 3D information relative to the camera (e.g., starting from an initial guess), until a satisfactory measure of similarity is achieved.

The first method may measure the similarity between a rendered image of a 3D model and an acquired image of the region of interest, where the 3D model that is used is that part of the model at the current stage of the procedure (e.g., it may be selected automatically or by a surgeon). For example, after craniotomy, the outer surface of the cortex may serve as such a model. The second method measures the similarity between the 3D model itself and a 3D model that is either derived from the stereoscopic image pair (e.g., based on the known calibration of the cameras) or derived from measurements by a 3D sensor (e.g., a TOF camera). An example of deriving the P&O of the 3D information in the camera coordinate system based on the second method includes matching the surface representation in the reference coordinate system with a corresponding surface in the 3D model by employing the “head and hat” method. Accordingly, a series of transformations which include homologous point matching is performed. In homologous point matching, each point in the hat (the surface representation) is associated with its nearest head point (3D model). A cost may be determined for each transformation. The transformation with the lowest cost may be determined as the transformation (e.g., the registration) between the surface representation and the 3D model.

As mentioned above, the above-described methods may rely on iteratively improving an estimated P&O of the 3D information relative to the camera, until a satisfactory measure of similarity is achieved. An initial guess of a relative P&O between the 3D information and the camera may be based on known initial orientation and distance between the camera and the imaged anatomy. For example, at the beginning of an endoscopic procedure the camera may move toward the region of interest from a generally known direction relative thereto. For example, in some laparoscopy procedures, the camera may typically enter from one side of the abdomen. In some endoscopic brain procedures, the camera may typically enter through the nose. These initial locations of the camera provide an initial guess of the initial orientation and distance between the camera and the region of interest. In some embodiments, the initial guess may be based on a registration and tracking as described earlier, which is then improved via, for instance, one of the two methods above, for example, to compensate for brain shift. In some embodiments, the P&O of the 3D information relative to the camera may be determined using different algorithms, such as ML/DL algorithms.

Following is an example relating to laparoscopy. During a laparoscopic liver biopsy procedure, a rendered image of a tumor may be superimposed on an image acquired by the laparoscope (e.g., an image of the outer surface of the liver). To that end, a conversion of locations between the 3D model of the liver (e.g., generated from an MRI scan) and the live image may be determined. Such a conversion of locations may be achieved by estimating the P&O of the 3D model with respect to the laparoscope (e.g., with respect to the camera, and assuming the camera is pre-calibrated), and iteratively improving the estimation based on a similarity measure as described above. To present a verification symbol relating to the validity of the conversion of locations, the surgeon marks on the 3D model, for example, blood vessels which are also visible on the surface of the liver in the live image. In some embodiments, verification elements are automatically identified by an algorithm. The verification symbol or symbols are then superimposed on the video image employing the conversion of locations.

Following is an example of using 3D information for guidance and for verification in ophthalmic surgery. A volumetric OCT scan (e.g., a 3D dataset comprising multiple OCT B-scans) of a retina may be acquired either preoperatively (e.g., using a diagnostic OCT device) or intraoperatively (e.g., using an intraoperative OCT). During a surgical procedure, guidance information that is generated based on the volumetric scan may be overlaid on an intraoperative image (e.g., a live image) of the surgical field. The guidance information may include, for example, an overlay that highlights areas of the retina having a membrane that needs to be peeled (e.g., the membrane may be automatically detected within the volumetric scan). To generate the overlay, the system registers the coordinate system of the volumetric scan with the coordinate system of the live image. For example, the registration may be based on a 2D image captured by the diagnostic OCT device concurrently with the volumetric OCT scan, along with information relating the location of each B-scan in the volumetric dataset with a line in the 2D image (e.g., information also provided by the diagnostic OCT device), and registering the 2D image with the live image.

In some embodiments, the registration may be based on generating a summed voxel projection (SVP) image from the volumetric scan and registering the SVP image with the live image. In some embodiments, when the volumetric scan is an intraoperative scan, the registration may be based, for example, on a known alignment between the OCT scanner and the camera that acquires the live image. In the examples above, to present a verification symbol relating to the validity of the conversion of locations, one or more image elements representing blood vessels, similar to the elements 316, 318 and 320 in FIG. 4B, are identified and located in either the SVP image or the 2D image. Thereafter, the same conversion of locations between coordinate systems used to generate the guidance information overlay may be employed to determine the location (or locations) associated with each of these image elements in the coordinate system of the live (e.g., intraoperative) image. Thereafter, a verification symbol respective of each one of the elements is superimposed on the intraoperative image.

FIG. 5 is a flow diagram of a method for providing a verification symbol relating to a validity of a conversion of locations between a first image of an eye of a patient (e.g., a preoperative image) and a second image of the eye of the patient (e.g., an intraoperative image), employed for ophthalmic surgery (e.g., as described above in FIGS. 1A, 1B and 1C), according to some embodiments of the invention.

The method may involve receiving a selection of an image element in the first image, the image element corresponding to a physical element in the second image. The physical element may be visible or assumed to be visible in the second image. The image element has a location in a first coordinate system, the first coordinate system being associated with the first image (Step 410).

In some embodiments, the selection of the image element is performed automatically by an algorithm (e.g., via computer 118, as described above in FIG. 1A). In some embodiments, the selection of the image elements is performed manually by a user via a user interface.

In some embodiments more than one image element is selected. Each image element represents a respective physical element. The at least one image element is visible (or assumed to be visible) in the second image. The at least one image element is associated with a location in a first coordinate system. The first image may be a 2D image of a region of interest, or 3D image information of the region of interest as described above. The physical element is, for example, a part of the anatomy, such as a blood vessel (e.g., scleral blood vessel, retinal blood vessel), a bifurcation point, a bone, an organ, a surface or contour of the physical element (e.g., the contour of the limbus), or a visible element on the iris (e.g., a spot). The physical element may be an artificial element (e.g., a fiducial marker and/or an implant). In some embodiments, the physical element corresponding to the selected image element employed for location conversion verification may be located at a location different from the location being operated on but within the FOV of the user. In some cases, the at least one image element is associated with multiple locations in the first coordinate system.

With reference to FIGS. 2A-2C, for example, bifurcation points 226, 228 and 230 are selected as the image elements in the first image, where preoperative image 200 is the first image. With reference to FIGS. 4A and 4B, for example, bifurcation points 303 and 305 and blood vessel 307 are as the image elements selected in the first image, where preoperative image 300 is the first image.

The method may involve determining (e.g., via computer 118 as described above in FIG. 1A) for the location in the first coordinate system, a corresponding location in a second coordinate system, which is associated with the second image, by employing the conversion of locations between the first coordinate system and the second coordinate system (Step 420).

The second image may be a 2D image (e.g., an intraoperative 2D image acquired by a microscope or an endoscope), or 3D image information of the region of interest as described above.

The conversion of locations between the first image and the second image may be achieved by registering the first coordinate system with the second coordinate system. In case of two 2D images the conversion of locations may be achieved, for example, by image registration. In some embodiments, the conversion of locations between coordinate systems of two 2D images may be achieved by employing corresponding anchor points in both coordinate systems, as described below. In the case of 3D guidance information, the conversion of locations may be achieved, for example, by registering the coordinates system of the 3D guidance information with the coordinate system of a tracker and further tracking the camera that acquires the intraoperative image (e.g., associated with the second coordinate system). In some cases, when the at least one image element is associated with multiple locations in the first coordinate system, corresponding multiple locations associated with the at least one image element are determined in a second coordinate system.

The method may involve displaying (e.g., via user display 102 and/or screen 108, as described above in FIGS. 1A-1C) the verification symbol superimposed with the second image based on the corresponding location in the second coordinate system (Step 430).

In some embodiments, the method may involve displaying guidance information on the second image. In the case of ophthalmic surgery, the guidance information may include, for example, information indicating a planned location and/or orientation of an intraocular lens, information indicating an actual location and/or orientation of an intraocular lens, information indicating a planned incision, information indicating a planned location and/or orientation of an implant (e.g., an implant for treating glaucoma), information relating to planned sub-retinal injection, information relating to a membrane removal, information indicating a location of an OCT scan, and/or information indicating a footprint of a field of view of an endoscope, or any other applicable information.

In some embodiments, the first image is intraoperative and the second image is preoperative. In some embodiments, the second image is intraoperative and the first image is preoperative.

In some embodiments, when the at least one image element is associated with multiple locations in the first coordinate system, a respective verification symbol is generated for the at least one image element and superimposed on the second image, based at least on the corresponding multiple locations.

When the conversion of locations between the first image and the second image is valid, the image element and corresponding verification symbol appear visually in alignment. When the conversion of locations between the first image and the second image is invalid, the image element and corresponding verification symbol appear visually out of alignment. The term ‘visually in alignment’ refers to the verification symbol directing toward the image element. The term ‘directing toward the image element’ includes pointing at the image element (e.g., in case an arrow is employed or a line with one end located at the corresponding converted location), at least partially encompassing the image element (e.g., in case of a geometrical shape or brackets are employed) or directly overlaying (e.g., when the verification symbol exhibits the shape of the physical element). The verification symbol is, for example, a frame (e.g., a circle, a rectangle) encompassing the image element (e.g., the limbus) or a wireframe model overlaid on the organ.

In some embodiments, the verification symbol is a cropped portion of a region of interest from a first image. The cropped portion may include the selected image element.

The description above relates to providing a visual indication relating to the validity of a conversion of locations (e.g., the validity of the conversion of locations is verified visually). According to some embodiments, the validity of a conversion of locations may also be verified automatically (e.g., by computer 118 of FIG. 1A). One example of automatically verifying the validity of conversion of locations may include identifying and selecting a region of interest in a first image (e.g., by employing trained neural networks), and determining a corresponding region in a second image using a conversion of locations. A similarity may be determined between the corresponding regions. For example, when the region is a polygon patch (e.g., rectangular), the matching locations of the vertices of the patch in the second image may be determined using the conversion of locations. The similarity between the patch in the first image and the patch in the second image (e.g., the patch specified by the four matching vertices locations) may be determined using various methods (e.g., correlation). Multiple regions of interest may be employed for automatic verification, so that the verification is robust to an occasional occlusion of some of the corresponding regions in the live image, for instance by a medical tool.

As mentioned above, some medical procedures entail the use of tracked medical tools. In some cases, the tool or tools are pre-fitted with a tracking unit, which enables tracking the P&O of the tool in a reference coordinate system. As described above, system 100 (FIGS. 1A-1C) may include a tracking system 103. Typically, the relationship between the coordinate system associated with tracking system 103 (e.g., a tool tracking coordinate system) and the coordinate system associated with the camera system 112 (e.g., “camera system coordinate system”), one with respect to the other, is known.

For example, in eye surgery systems (e.g., ophthalmic surgery systems), a camera (e.g., which acquires a live image) and a tracking imager (e.g., which tracks the tool tracking unit) may both be mounted on the same frame and the spatial relationship therebetween may be pre-calibrated.

In another example, in a brain or spine surgery system, each one of a camera, a tool and an HMD (if employed) may be tracked relative to a tracker reference unit attached to the patient. The spatial relationship between the camera and the tool may be tracked (e.g., repeatedly determined) relative to each other.

In another example, tracked tools may also be used in visor-guided surgery (VGS) procedures. In VGS procedures, an HMD may augment a surgeon's view of a patient and may allow the surgeon to see anatomical features and/or surgical tools as if the patient's body were partially transparent. These procedures may optionally be performed entirely without a magnified image of the surgical field and therefore without a camera head unit. Nevertheless, the HMD may comprise a camera or cameras for various functions. The HMD may also include a tracking unit, and the tracking system may repeatedly determine relative P&Os between the HMD tracking unit, a patient tracking unit, and a tool tracking unit.

The tracking units may be optical, electromagnetic and/or other type of tracking units as are known in the art. The tracking units may include one or more sensors, one or more cameras, one or more markers, or any combination thereof. Markers may be, for example, ARUCO markers or light reflectors (e.g., passive markers) and/or LEDs (e.g., active markers). The spatial relationship between the HMD tracker unit and the camera (or cameras) is typically pre-calibrated and known, hence the spatial relationship between the camera and the tool may be tracked. In some cases, tools are not pre-fitted with tool tracking units. Nevertheless, when tools are not pre-fitted with tool tracking units, such tool tracking units may be attached to such tools to provide tracking capabilities to these tools. However, the spatial relationship between the tool tracking unit and the tool is unknown and may be determined. Determining the spatial relationship between a tracked unit and a tool to which the tracked unit is attached, e.g., tool alignment.

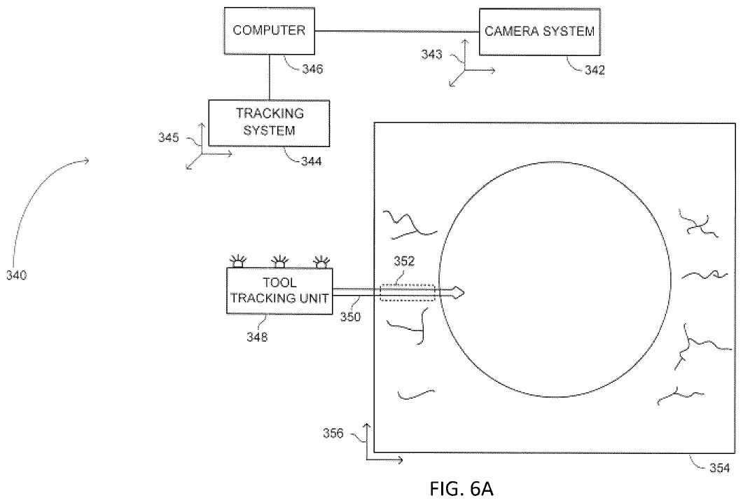

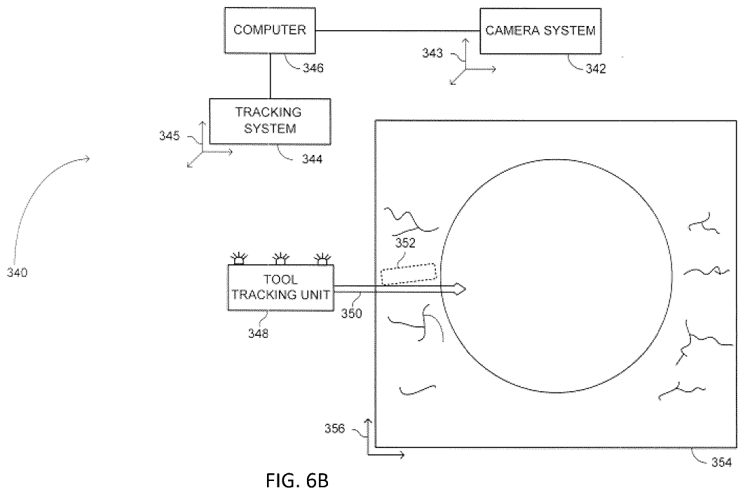

FIGS. 6A and 6B are schematic diagrams of a system 340 for tool alignment, according to some embodiments of the invention.

System 340 includes a camera system 342, a tracking system 344 and a computer 346. Tracking system 344 tracks a tool tracking unit 348 attached to tool 350. Computer 346 is coupled with camera system 342 and with tracking system 344. Camera system 342 may be a stereoscopic camera which acquires a stereoscopic image pair (e.g., employed in a microsurgical procedure). Camera system 342 may be associated with a camera coordinate system 343 and tracking system 344 may be associated with a tracking coordinate system 345. Tracking system 344 may track tool tracking unit 348 in tracking coordinate system 345.

Tracking system 344 may measure a P&O of tool tracking unit 348 in tracking coordinate system 345. Camera coordinate system 343 and tracking coordinate system 345 may be pre-registered one with respect to the other. In the description which follows, the example of a stereoscopic camera which produces a stereoscopic image pair is employed. However, the alignment method described herein may also be employed using only one camera (e.g., when the camera system comprises a single camera).

Tool tracking unit 348 may be attached to tool 350. The spatial relationship between tool 350 and tool tracking unit 348 may be unknown (e.g., to a selected degree of accuracy). In some embodiments, to determine the spatial relationship between tool tracking unit 348 and tool 350, once tool tracking unit 348 is attached to tool 350, the user may move tool 350 into the FOV of camera system 342. Tracking system 344 may acquire a respective P&O measurement of tool tracking unit 348 in tracking coordinate system 345, to determine one or more tool tracking unit measured P&Os. The camera system 342 may acquire a stereoscopic image pair of tool 350, such that each image pair is associated with a respective measured P&O of tool tracking unit 348 (e.g., by employing synchronous acquisitions of stereoscopic image pairs and measured P&Os, and/or by employing time-stamps).

For example, for each measured tool tracking unit P&O in tracking coordinate system 345, computer 346 may determine a respective tool tracking unit P&O in camera coordinate system 343, based on the pre-registration between camera coordinate system 343 and tracking coordinate system 345. Based on the tool tracking unit P&O in camera coordinate system 343, an estimate (e.g., an initial guess) of the tool alignment, and a stored 3D model of tool 350, computer 346 renders two images of the stored 3D model of tool 350, from each of the two points-of-view (POVs) of the two cameras of the stereoscopic imager. The 3D tool model may include only portions of the tool that are assumed to exhibit a direct line of sight with the camera (e.g., without the tool's handle, which is assumed to be hidden by the user's hand). Each measured tool tracking unit P&O may be associated with a pair of rendered images of the 3D tool model. When the estimated tool alignment is identical to the actual tool alignment, the location and orientation of the tool model, in each of these two rendered images, is identical to the location and orientation of the actual tool in each of the corresponding acquired stereoscopic image pair of the actual tool.

In general, cameras may exhibit optical distortions that typically are pre-calibrated. Computer 346 may correct the acquired stereoscopic image pair to account for these distortions. In some embodiments, for instance when the distortions in the acquired images are not corrected, computer 346 may distort the rendered images, such that when the estimated tool alignment is identical to the actual tool alignment, the location and orientation of the tool as it appears in the rendered images and in the images of the actual tool are identical.

In some embodiments, the computer 346 determines a tool alignment which optimizes (e.g., minimizes or maximizes) a cost function (e.g., by employing the Newton-Raphson method), where the cost function is based on a similarity score. For example, computer 346 determines similarity scores between each acquired image and its respective rendered image and determines the value of the cost function based on these scores. Each estimated tool alignment is associated with a value of a cost function. Computer 346 repeatedly (e.g., iteratively), re-estimates the tool alignment as described above, until a satisfactory value of the cost function is obtained or after a determined number of iterations. For example, when the value of the cost function is not satisfactory (e.g., higher than a pre-defined threshold), computer 346 determines the change or changes required to the estimated alignment, in one, some or all of 6 DOF (e.g., by employing globally convergent methods), which best improves the value of the cost function. In some embodiments, prior to determining a similarity score, computer 346 may pre-process the acquired and/or the rendered image (e.g., by performing segmentation to identify tool 350 in the acquired image). In these embodiments, a single camera may be used. In various embodiments, the tool alignment may be achieved via machine learning or deep learning networks.

In some embodiments, the tool alignment is based on the measured tool tracker P&Os and acquired corresponding stereoscopic image pairs. In this embodiment, camera system 342 includes two cameras. In some embodiments, a 3D sensor may be employed, as further described below. Computer 346 may generate an actual 3D model of tool 350 in camera coordinate system 343, from the stereoscopic image pair (e.g., a reconstructed 3D model of the surface of the tool as seen in the stereoscopic image pair). This reconstructed 3D tool model of tool 350 may be based on a 3D map of the scene in the stereoscopic image pair, from known camera-system calibration. The camera-system calibration may include the pre-calibrated spatial relationship between the two cameras of the stereoscopic imager, and (for each camera) the camera mapping that determines a transformation between locations in images acquired by the camera and corresponding directions in the camera coordinate system. The reconstruction of the 3D tool model employs, for example, triangulation of image elements in both images that are identified to be identical. A 3D map of the scene may be based on a 3D sensor in camera system 342, such as a time-of-flight sensor or a structured light sensor. In a VGS system without a camera head unit, the 3D sensor may be embedded, facing forward, in the HMD.

Employing the stored 3D model of tool 350, computer 346 determines a P&O of the reconstructed 3D tool model in the camera system coordinate system 343 (e.g., by comparing the stored and reconstructed models). Computer 346 also determines the P&O of the reconstructed 3D tool model in tracking coordinate system 345. Computer 346 may then determine the tool alignment that transforms a measured tool tracker P&O to the P&O of the reconstructed 3D model (e.g., in tracking coordinate system 345). In some embodiments, computer 346 may determine the P&O of the stored 3D model in camera system coordinate system 343 and determine the tool alignment that transforms a measured tool tracker P&O to the P&O of the reconstructed 3D model in camera system coordinate system 343. This process may be repeated for each set of measured tool tracker P&O and corresponding acquired images, and an average tool alignment may be determined as the final tool alignment.

Irrespective of how the spatial relationship between tool tracking unit 348 and tool 350 (e.g., the tool alignment) is determined, the user may be provided with a visual indication relating to the validity of the alignment. A tool alignment verification symbol 352 corresponding to tool 350, e.g., tool symbol, may be generated on an image 354, and presented to the user.

Image 354 may be one of a stereoscopic image pair acquired by camera system 342. Image 354 is associated with an image coordinate system 356. Computer 346 generates tool symbol 352 and may overlay tool symbol 352 on image 354, according to the P&O of tool tracking unit 348 in image coordinate system 356 and the determined tool alignment. To overlay tool symbol 352, computer 346 may determine the P&O of a tool model in tracking coordinate system 345 based on the P&O of tool tracking unit 348 and the tool alignment. The computer 346 may determine the P&O of the tool model in camera coordinate system 343, based on the known spatial relationship between tracking coordinate system 345 and camera coordinate system 343.

In some embodiments, computer 346 renders an image of the tool model from the POV of the camera and overlays the rendered image as tool symbol 352 on image 360. In some embodiments, computer 346 renders a model associated with the tool, for example, a tool envelope (e.g., a cylinder enveloping an elongated part of the tool, as depicted by symbol 352), having the same coordinate system as the tool. If the optical distortions of camera system 342 are not corrected when displaying image 354 to the user, computer 346 may distort the rendered image of the tool model (or the model associated with the tool) before overlaying the tool model as tool symbol 352 on an acquired image.

When tool 350 and tool symbol 352 appear visually in alignment, then the tool alignment is valid. When tool 350 and tool symbol 352 appear visually out of alignment, then the tool alignment is invalid.

In FIGS. 6A-6B, tool symbol 352 is depicted as a rectangle, representing a cylinder that in 3D appears to encircle the elongated part of tool 350 when viewed using a stereoscopic display. In some embodiments, tool symbol 352 may be a line positioned along the axis of tool 350.

In some embodiments, tool symbol 352 is generated on both images of a stereoscopic image pair which is presented to a user, thereby providing the user with a 3D perspective of tool 350 as well as of tool symbol 352. Tool symbol 352 may also exhibit, for example, the shape of a series of rings or squares in the 3D space, centered along the axis of tool 350 or a combination of such rings or squares and a line positioned along the axis of tool 350. As a further example, the tool symbol may be a 3D model of the tool, for example, when the tool is rotationally asymmetric (e.g., a curved tool). The model pertinent to the tool being used may be selected from a user interface. The model displayed to the user may be, for example, a wireframe model overlaid such that both the tool and the tool model are visible to the user.

The visual indication may also be employed during the process of acquiring the data for alignment of the tool tracking unit with the tool, thus providing the user with an indication regarding the progress of the alignment process. For example, while additional datasets including the tool tracker P&O and corresponding images are being acquired (e.g., as the user moves the tool under the camera), the computer may periodically calculate updated estimates of the tool alignment. The user may choose to stop the alignment process before the end thereof if the user decides that the accuracy of the current alignment estimate (e.g., as manifested by the visually aligned appearance of the tool and the tool symbol) is sufficient.

In some embodiments, during the alignment process, system 300 may provide the user with information relating to the alignment process. This alignment process information includes, for example, the current alignment phase (e.g., “gathering data”, “calculating”, “calculation completed”), current alignment error, the current iteration number or the current value of the cost function. Alignment process information may further include instructions to the user to carry out actions such as “rotate tool” or “move tool to the left”.

In some embodiments, the system may have effective misalignment. Possible causes of effective misalignment may be: i) wrong tool alignment, ii) tracking problems, iii) HMD movement relative to the user's head that occurred after eye location calibration (e.g., when an optical see-though HMD is utilized, such as in VGS systems), and/or iv) tool deformation. When tool 350 and tool symbol 352 appear visually out of alignment, then the effective tool alignment is invalid, for instance due to accidental movement of tool tracking unit 348 relative to tool 350.

A tracking problem may be caused, for example, by droplets of liquid on tool tracking unit 348 (e.g., when the tracker is an optical tracker), by an electro-magnetic interference (e.g., when the tracker is an electro-magnetic tracker), and/or by accidental movement of a tracking reference unit of tracking system 344 relative to camera system 342. In brain and spine surgery, for example, the cause of such a misalignment may also be accidental movement of the tracking reference unit or droplets of blood on the tracking reference unit. As another example, tool symbol 352 appearing visually out of alignment may be indicative of the HMD moving relative the user's head since the location of the HMD was calibrated at the beginning of the procedure (e.g., when the HMD does not comprise means for tracking the user's eyes and compensating for such relative movement). In another example, the tool symbol 352 may appear visually out of alignment because of a smudge on optics of a sensor or an optical tracking unit embedded in the HMD. In such cases, the system may guide the user in identifying the source of the misalignment.

The system may guide the user in identifying a source of the misalignment in systems with or without a camera. In some embodiments, when the system has a
a camera system, the system may automatically identify a wrong tool alignment (e.g., by automatically re-determining the tool alignment), a deformed tool (e.g., by comparing an image or images of the tool to a stored 3D model of the tool), and a tracking problem (that may likely lead to failure in automatically determining the tool alignment). If all of these possible causes are determined not to be the cause of the misalignment, the system may guide the user to re-calibrate the eyes locations.

In some embodiments, for a system without a camera system, the system may guide the user through a process of identifying and correcting a state of misalignment. For instance, if the user identifies a misalignment (e.g., with tool no. 1), the system may suggest that the user checks another tool (e.g., tool no. 2). If the verification symbol for tool 2 is properly aligned with tool 2 then the HMD may be excluded as the cause for misalignment of tool 1, and the source of the misalignment of tool 1 may be a wrong alignment between tool 1 and the tracking unit attached to it (tracking unit 1), a problem with tracking unit 1 (e.g., droplets of blood on a marker (LED, reflective sphere, etc.) of tracking unit 1, or tool 1 being deformed. The system may then suggest that the user cleans tracking unit 1. If the problem persists, the system may offer to check if the tool is deformed, for instance by holding it against another tool (e.g., when both tools are supposed to be straight). The system may also suggest re-calibrating the tool 1 alignment by using a dedicated jig. If the verification symbol for tool 2 is not properly aligned with tool 2 (e.g., it is misaligned) then the system may guide the user to clean the HMD tracking unit (or units), and if the problem persists to re-calibrate the eyes locations. In various embodiments, the order of suggestions made by the system to direct the user through the process may vary based on particular components of the system.

In some embodiments, system 300 may periodically and automatically check the validity of the alignment and display a warning and instructions if a misalignment is detected. This may be achieved, for example, by periodically saving images (or 3D data when a 3D sensor is used instead of a camera) and corresponding tool tracking unit P&O values and determining the similarity score as described above between the saved images and corresponding rendered images of the tool model. In some embodiments when the validity of the alignment is automatically determined, instead of displaying the verification symbol the system may display an indication regarding the validity of the alignment (e.g., as a red or green flag at the corner of the display), as automatically determined. In some embodiments when the validity of the alignment is automatically determined, an indication regarding the validity of the alignment is displayed only when misalignment is detected (e.g., as a red flag, or as a warning message). In some embodiments when the validity of the alignment is automatically determined, the verification symbol is displayed only when misalignment is detected.

FIG. 7 is a flow diagram of a method for providing a verification symbol relating to a validity of an effective alignment of a tool tracking unit (e.g., tool tracking unit 348 as described above in FIGS. 6A and 6B) with a medical tool (e.g., tool 350 as described above in FIGS. 6A and 6B) employed in a medical procedure, according to some embodiments of the invention.

The method involves determining a tool alignment, or receiving a predetermined tool alignment, between the medical tool and the tool tracking unit (Step 610). For example, a first P&O of the tool in a reference coordinate system may be determined from an acquired stereoscopic image of the tool. A second P&O of the tool in a reference coordinate system may be determined by a tracking system. The alignment between the tool and the tool tracking unit may be done as described above with respect to FIGS. 5A and 5B.

The method involves receiving information relating to a geometry of the medical tool (Step 620). The information related to geometry may be 3D model of the medical tool, a line, or other geometrical representations. For example, an elongated tool may be represented as a line, with or without the diameter of the line. When the tool is, for example, a tool used for placing a pedicle screw, the model may include a model of the screw that is attached to the tip of the tool, specifically including its diameter and length. In general, when the tool is used for placing an implant, the model may include the implant.

The method involves generating the verification symbol based on the tool alignment and the information relating to the geometry of the medical tool (Step 630).

The verification symbol may be generated at least according to the determined tool alignment. The verification symbol may be generated on a live image and presented to the user (e.g., overlaid on the live image). For example, the verification symbol may be an image of a tool model rendered from the POV of the camera. When the tool and the tool symbol appear visually in alignment, then the tool alignment is valid. When the tool and the tool symbol appear visually out of alignment, then the tool alignment is invalid. The verification symbol is overlaid on the live image based on the P&O of a tracked tool tracking unit in the camera coordinate system and the P&O of the tool model in the camera coordinate system, as explained above in conjunction with FIGS. 5A and 5B.

In some embodiments, additional tool alignment information may be presented to the user during tool alignment. The tool alignment information may include alignment process information, relating to the alignment process itself. The alignment process information may include the current alignment phase (e.g., “gathering data”, “calculating”, “calculation completed”), current alignment error, the current iteration number or the current value of the cost function. Alignment process information may further include instructions to the user to carry out actions such as “rotate tool” or “move tool to the left”.

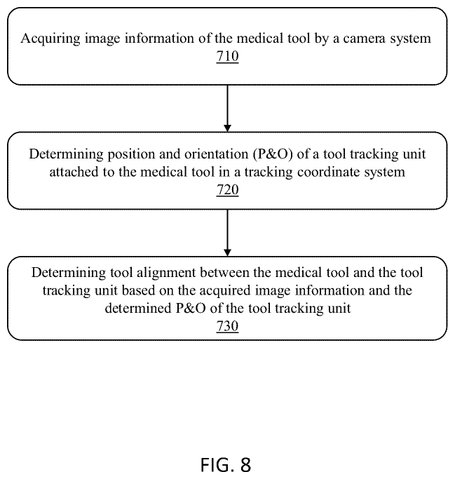

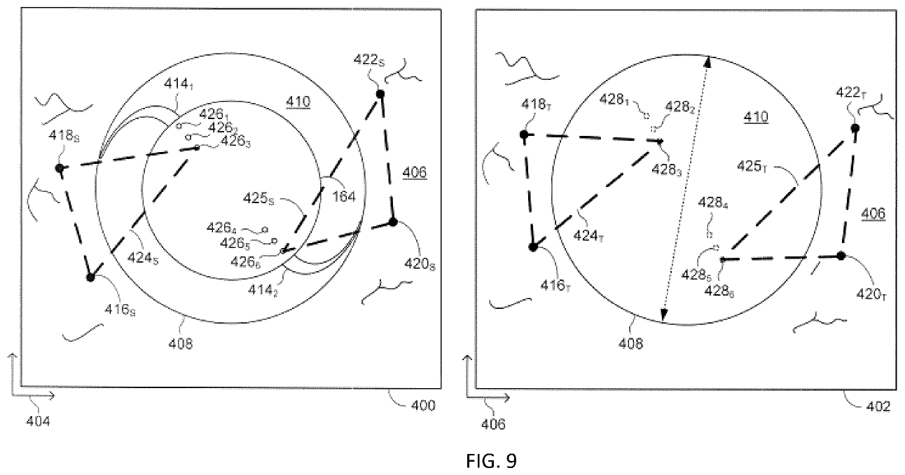

FIG. 8 is a flow diagram of a method for determining alignment of a tool tracking unit with a medical tool employed in a medical procedure, according to some embodiments of the invention.

The method involves acquiring image information of the medical tool by a camera system (e.g., via camera system 342 as described above in FIGS. 6A and 6B) (Step 710). The acquired image information may be a single 2D image or a stereoscopic image pair.

The method involves determining a position and orientation (P&O) of a tool tracking unit attached to the medical tool in a tracking coordinate system (Step 720). For example, tracking system 344 may determine the P&O of tool tracking unit 348 in tracking coordinate system 345 as described above in FIGS. 6A and 6B.

The method involves determining (e.g., via computer 346 as described above in FIGS. 6A and 6B) tool alignment between the medical tool and the tool tracking unit, based on the acquired image information and the determined P&O of the tool tracking unit (Step 730). The alignment between the tool and the tool tracker unit may be determined based on the image information of the tool and the position and orientation of the tool tracker unit. The alignment between the tool and the tool tracker unit may be determined according to any one of the examples described above in conjunction with FIGS. 6A and 6B as described above.

The tool alignment verification symbol may be generated regardless of the alignment process being employed and/or whether or not the system includes a camera system. For example, the tool alignment may be determined according to the method described herein above in conjunction with FIG. 8, employing a jig as described above, or according to any other technique which produces information relating to the alignment between the tool and the tool tracker. The tool may also be pre-fitted with a tool tracking unit and the alignment information provided with the tool. In either case, a tool alignment verification symbol may be generated and displayed to allow the user to verify that the tool alignment is valid.

In some embodiments, the methods described in FIGS. 7 and 8 are implemented with a system comprising an HMD with an embedded camera system. In these embodiments, the relative P&O between the HMD and the tool may be tracked or determined. The relative P&O may be directly tracked, e.g., when the tool tracker unit is directly tracked by the HMD tracker unit or units, and/or vice versa. In some embodiments, the relative P&O may be determined by separately tracking the P&O of the HMD and the P&O of the tool in a common reference coordinate system. Such a system may be, for example, a VGS system as described above. In these embodiments, the camera (or cameras) or the 3D sensor in the HMD camera system may be utilized for determining the alignment between the tool tracker unit and the tool (e.g., as described with respect to FIGS. 6-8 above). Tracking the tool may be based on a tracking component embedded in, or attached to, both the HMD and the tool. For example, a tracking unit in the HMD may include a camera and an LED (or LEDs) and may track reflectors of a tool tracker unit that reflect light from the LED and may also track a patient tracker unit that is rigidly attached to a patient. In some embodiments, a camera or cameras outside the surgical field may track tracker units attached to each of the patient, the tool, and the HMD. Independent of how tracking is implemented, the camera system may be used for tool alignment and/or tool alignment verification.

In some embodiments, automatic tool identification may be employed. In these embodiments, when the system has a database of 3D models of possible tools, the system may automatically identify a tool based on the acquired image or images (or acquired 3D model). This may be implemented by machine learning or deep learning networks that were trained to identify a tool type based on an acquired image or images (or based on an acquired 3D model) of the tool, or by any algorithm known in the art.

When the interface between the tool and the tool tracker guarantees an accurate and known alignment (e.g., per tool type), once the tool type is identified (e.g., either automatically or manually by user selection) the system may determine the tool alignment (e.g., as described above with respect to FIG. 8), compare the determined alignment with the known alignment, and alert if they do not concur.

In some embodiments, the system may display the tool alignment verification symbol (e.g., based on the known alignment), thus allowing the user to verify that the pre-determined known alignment was correctly read from a memory and/or that the tool tracker was correctly attached to the tool (e.g., in addition to verifying that the tracking is accurate, that the tool is not mechanically deformed, and that the HMD has not moved relative to the head of the user). When the alignment is unknown, the system may identify the tool type, determine the alignment (e.g., as described above with respect to FIG. 8), and display the tool alignment verification symbol (e.g., based on the determined alignment) to allow the user to verify the alignment.

In some embodiments, the invention involves a method for automatically identifying and alerting when a tool is mechanically deformed (e.g., after multiple uses, the shape of a tool may be changed. For example, orthopedic surgeons may use force that may cause a tool that was originally straight to become curved). Navigated procedures rely on the shape of the tool for accurate guidance, therefore a deformed tool may negatively affect the outcome of the procedure. When the system identifies that a tool is deformed it may alert the surgeon and recommend that the tool is replaced. Automatically identifying a deformed tool may be implemented, for example, by first identifying the tool type (e.g., automatically as described above, or manually by user selection), and then comparing the acquired image or images (or the acquired 3D model) to the 3D model of the tool from the database of 3D models of possible tools to detect mechanical deviations in the tool. In some embodiments, algorithms for identifying the tool type may identify the tool type even when the tool is deformed.

Both automatic tool identification and automatically identifying and alerting when a tool is mechanically deformed may be implemented either by a system with a camera head unit, as shown, for example, in FIG. 1C, or by a system with a camera system embedded in an HMD. Overlaying the tool alignment verification symbol may be done either when the user is viewing an image of the surgical field (e.g., live video), and the overlay is superimposed with the live video that the user is viewing, or when the user is directly viewing the surgical field through an optical see-through HMD and the overlay is superimposed with the view of the actual tool via the HMD optics. In some embodiments, the methods above may be implemented with a display system that allows directly viewing the surgical field through a semi-transparent display that is not head-mounted.

As described above, tool alignment may be determined and/or verified with a tracked jig (or a tracked pointer), for instance when using a VGS system where the HMD does not comprise a camera system, or when the HMD does comprise a camera system but the user prefers to determine and/or verify the tool alignment with the help of a tracked jig or pointer. In some embodiments, in addition to employing the jig, the system may also display the alignment verification symbol via the HMD, thus providing the user with visual feedback regarding the validity and the accuracy of the alignment. In some embodiments, the system may also provide the user with a jig verification symbol. The jig verification symbol may be generated based on the known 3D model of the jig and the known alignment between the jig tracker unit and the jig, similar to the method for generating the tool alignment verification symbol (e.g., the tool alignment verification symbol is overlaid with the tool, whereas the jig verification symbol is overlaid with the jig). The jig verification symbol may not be required for providing the user with an indication of the accuracy of the alignment between the jig tracker unit and the jig, since the jig is pre-fitted with a tracker unit and pre-calibrated (e.g., factory-calibrated), and assumed to be accurate. Rather, the jig verification symbol may be used for calibrating the locations of the user's eyes relative to the HMD, as described below. The same may be done with a tracked pointer that is pre-fitted and pre-calibrated, as described above with respect to tool alignment with a jig or a pointer. When using a pointer, a pointer verification symbol may be used for calibrating the locations of the user's eyes relative to the HMD. In general, any tool may be used with a corresponding tool verification symbol for calibrating the locations of the user's eyes relative to the HMD.

As described earlier, during VGS procedures the surgeon may see representations of the patient's anatomy and additional guidance data, superimposed and accurately registered to the patient's body. When generating the overlay image (or images, for a stereoscopic HMD) that the surgeon sees via the optical see-through HMD, the system may take into consideration the location of the surgeon's eye (or eyes) relative to the HMD optics. This may be important for procedures in which high accuracy is required. In some cases, the HMD shape guarantees that the eye locations are known, by guaranteeing that each time the surgeon dons the HMD the eyes are located at the same known or pre-calibrated location relative to the HMD optics. In other cases, the HMD may comprise an eye tracker that may provide the eye location. In some embodiments, the system may require that the eyes' locations are calibrated after the surgeon dons the HMD and before the procedure (e.g., before the step of the procedure that requires high accuracy).

The calibration process may be based on adjusting the xyz values of the locations of the user's eyes (e.g., in the HMD coordinate system) that are employed by the system to generate the overlay images, such that the jig and the jig verification symbol are correctly aligned. When the xyz values are adjusted, the 3D location of the jig verification symbol, as seen by the user, may be changed, and the user may continue the adjustment until the jig and the jig verification symbol are correctly aligned.

Controlling the adjustment may be done in several ways. Note that the locations of both eyes may be adjusted concurrently, since typically in HMDs the z (e.g., up-down) and x (e.g., forward-backward) locations of the left and right eyes relative to the display optics are identical, and the y location is changed in opposite directions (e.g., as the eyes are symmetrically located relative to the nose). In some embodiments, controlling the xyz values is adjusting the values via a touchscreen, a keyboard, or by employing an HMD menu. In some embodiments, the xyz values are adjusted by head gestures. For example, an up-down head gesture (a “yes” gesture) may control the z value, a left right gesture (a “no” gesture) may control the y values, and forward-backward head movement may control the x value.

All motions (e.g., gestures) may be relative to a fixed coordinate system or relative to the tracked jig. In some embodiments, if the jig is a handheld jig, the user may move the jig in up-down, left-right and forward-backward movements (e.g., relative to the HMD), to adjust the z, y and x values of the eyes' locations respectively. The adjustment may be enabled, for instance, by pressing a footswitch, by pressing a button embedded in the jig or attached to it, by voice command, and so on (e.g., the xyz values are updated only while the user enables the adjustment).

The same methods for adjusting the xyz values that the system uses for generating the overlay images based on aligning a jig verification symbol with a jig (or a pointer verification with a pointer) may be similarly used for adjusting any value that the system uses when generating the overlay images and that may differ between different users or for different occasions the HMD is used by the same user.

As mentioned above herein, conversion of locations may relate to determining a location in a second coordinate system (e.g., associated with a second image), which corresponds to a location in a first coordinate system (e.g., associated with a first image), or vice versa (e.g., determining a location in the first coordinate system, which corresponds to a location in the second coordinate system). Further, as mentioned above, some embodiments relate to verifying the validity of overlaid guidance information. The guidance information is defined in one coordinate system (e.g., a 2D coordinate system of an image, a 3D coordinate system of a 3D dataset) and overlaid on an image associated with another coordinate system, employing a conversion of locations. The same conversion of locations employed to overlay the guidance information is also employed to generate and display a verification symbol for visually verifying the validity of the conversion of locations (e.g., the validity of the overlaid guidance information).

In some embodiments, for guidance information defined with respect to a 3D coordinate system, the conversion of locations between the 3D coordinate system and the 2D coordinate system of an intraoperative image is based on registration of the 3D coordinate system with a tracking coordinate system and tracking the camera that acquires the intraoperative image. In these embodiments, the same registration and the same tracking information for generating both the guidance overlay and the verification symbol may be used. The conversion of locations may be employed to convert locations from the 3D coordinate system of the 3D information to the 2D coordinate system of the intraoperative image or vice versa.

In some embodiments for guidance information defined with respect to a 3D coordinate system, the conversion of locations between the 3D coordinate system and the 2D coordinate system of an intraoperative image is based on generating a 2D image from the 3D dataset (e.g., an SVP image generated from a volumetric OCT scan) and aligning the 2D image to the intraoperative image (e.g., performing image registration between the SVP image and the live image). In these embodiments, using the same transformation between the 3D coordinate system of the 3D dataset and the 2D coordinate system of an intraoperative image for generating both the guidance overlay and the verification symbol may be used. In general, any method for conversion of locations may be used for conversion of locations between the 3D coordinate system and the 2D coordinate system (and vice versa) as long as the same conversion is used for generating both the guidance overlay and the verification symbol.

Following are two examples of conversion of locations between two 2D images. As mentioned above, one example of conversion of locations between coordinate systems of two 2D images may be based on registering the coordinate systems. Another example of conversion of locations between coordinate systems includes converting selected locations between the coordinate systems (e.g., without performing registration). In both examples, as noted above, the conversion of locations may be employed from one coordinate system to the other coordinate system and vice versa. In both of the above examples, the first stage of conversions of locations between coordinate systems is identifying image features (or simply “features”) in both images and finding pairs of matching features, where each pair consists of one feature in each image. Each feature has a well-defined location in the corresponding image thereof. Matching feature pairs are assumed to represent the same point in the scene.

As mentioned above, identifying features may be achieved by image processing techniques such as Scale Invariant Feature Transform (SIFT) or Speeded Up Robust Features (SURF), or other techniques such as deep learning. Identifying a feature in an image may include identifying an image region and providing a descriptor or descriptors of this region. The descriptors may include, for example, information relating to color, texture, shape and/or location. Once features are identified in both images, these features may be paired according to similarity between their respective descriptors.

When employing image registration for conversion of locations between two images, a single mathematical transformation, f(x, y)→(x′, y′) may be determined between the first image and the second image to convert locations between the first image and the second image, where (x, y) relates to the location in the first image and (x′, y′) relates to the location in the second image, where x, y, x′ and y′ are in units of pixels of the respective image and may have non-integer values. The resolution of the first image and the second image need not be the same resolution. The inverse of the transformation f(x, y) (e.g., f-1(x′, y′)→(x, y)) may be employed to convert locations between the second image and the first image. In this example, guidance information defined with respect to a first coordinate system associated with a first image is overlaid on a second image associated with a second coordinate system, based on a transformation f(x, y). A verification symbol, corresponding to an image element in the first image, is overlaid on the second image based on the transformation f(x, y). In some embodiments, a verification symbol, corresponding to an image element in the second image is overlaid on the first image based on the transformation f-1(x′, y′). In this example, the term “same conversion of locations” may refer to using either f(x, y) or f-1(x′, y′) or both for generating a verification symbol when the guidance information is generated based on f(x, y).

According to another example of conversion of locations between two 2D images, locations are converted between coordinate systems associated with the two images without determining a mathematical transformation between the coordinate systems, e.g., no registration of the images is required. According to this example, a set of pairs of matching features (e.g., feature-pairs) in the two images are determined as described above with respect to the image registration example. When converting a location (e.g., a point of interest) in the first image to a corresponding location in the second image, two feature-pairs from the set of feature-pairs are selected. Each feature-pair includes a feature in the first image and a matching feature in the second image. The locations of the two features in the first image and the location of the point of interest in the first image define a virtual triangle (e.g., the two features and the point of interest are the vertices of the triangle). A similar triangle is constructed (e.g., virtually) in the second image, employing the matching (e.g., paired) features in the second image, thus defining the corresponding location of the point of interest in the second image. In other words, two features, for example, located at location A and location B respectively, are selected in the first image, these features having two matching features, at respective locations A′ and B′ in the second image. Thereafter, for location C of the point of interest in the first image, a corresponding location C′ is determined in the second image, such that triangle A′B′C′ is similar to triangle ABC.

In this example, guidance information defined with respect to a first coordinate system associated with a first image is overlaid on a second image associated with a second coordinate system, based on similar triangles as described above. For example, a line determined in the first coordinate system is represented as two points (e.g., the two edges of the line) and the locations of these two points are converted to the second coordinate system to define a corresponding line in the second coordinate system. The locations of these points may be converted using similar triangles as described above. The location of each point is converted using a triangle in the first image which is constructed by selecting two feature-pairs from the set of feature-pairs and constructing a triangle in the second image such that the triangle in the second image is similar to the triangle in the first image. A verification symbol, corresponding to an image element in the first image, is overlaid on the second image based on the same set of feature-pairs. The verification symbol may be represented by points. For example, a bifurcation of a blood vessel may be represented by four points. The location of each point of the verification symbol is converted using a triangle in the first image which is constructed by selecting two feature-pairs from the set of feature-pairs and constructing a triangle in the second image such that the triangle in the second image is similar to the triangle in the first image. In some embodiments, a verification symbol, corresponding to an image element in the second image is overlaid on the first image based on the same set of feature-pairs. The assumed location of the image element in the first image is determined based on constructing triangles in the second image and determining triangles in the first image, such that the triangles in the first image are similar to their respective triangles in the second image. In this example, the term “same conversion of locations” referred to hereinabove, relates to using the same set of feature-pairs for generating both the guidance information and the verification symbol.

Typically, multiple couples of features located at locations [A1, B1]-[AN, BN] are selected in the first image, and multiple triangles are constructed using these feature locations and the location C of the point of interest. The similar triangles [A′1B′1C′1]-[A′NB′NC′N] in the second image define multiple locations (e.g., C′1-C′N), which are averaged to generate the converted location C′ of the point of interest. When converting locations from the second image to the first image, the same set of feature-pairs is used, from which multiple selected couples of feature-pairs are employed for the conversion of locations.

FIG. 8 is a schematic illustration of a conversion of the position of points of interest from a source image 400 to a target image 402, according to some embodiments of the invention.

Source image 400 is associated with a source coordinate system 404 and target image 402 is associated with a target coordinate system 406. Source image 400 is an intraoperative image of an eye, during placement of a toric Intraocular Lens (IOL), such as toric IOL 212 as described above with respect to FIG. 2A and target image 402 is a preoperative image of the eye. Target image 402 and source image 400 are images of the same scene. However, target image 402 is rotated relative to source image 400. Toric IOL 164 includes two haptics 4141 and 4142 intended to hold toric IOL 164 in place, once toric IOL 164 is correctly positioned. Furthermore, toric IOL 164 includes axis marks 4261, 4262, 4263, 4264, 4265 and 4266. Presented in source image 400 and in target image 402 are the sclera 406, the pupil 410 and various blood vessels. In the example brought forth in FIG. 8, the points of interest are axis marks 4263 4266 and it is required to determine the location thereof in target coordinate system 406.

Initially, at least two pairs of features are identified in source image 400 and target image 402. These pairs of features are identified by detecting features in both images and matching features to generate feature pairs. For example, in FIG. 8, features 416T, 418T, 420T, 422T are selected in target image 402 with matching features 416S, 418S, 420S, 422S. In FIG. 8, four feature-pairs are brought forth as an example. However, in general, at least two feature-pairs are required and typically, tens and even hundreds of feature-pairs may be identified. It is noted that features 416T, 418T, 420T, 422T and 416S, 418S, 420S, 422S are not necessarily associated with prominent visible elements in the images. Typically, the features are selected automatically as described above and not by a user. In the location conversion method described herein, the feature-pairs may be determined once for each pair of images. In the case where one of the images is a live image, once features are determined they may be tracked in the live image and their locations may be continuously updated. Different features may be selected every predetermined time period or based on changes in the live image.

To convert the location of axis mark 4263 from source coordinate system 404 to target coordinate system 406, features 416S, 418S are selected. Features 416S, 418S and axis mark 4263 define a triangle in source coordinate system 404, where a line defined by features 416S, 418S forms a side of triangle 424S. Similarly, features 420S, 422S and axis mark 4266 define a triangle 425s in source coordinate system 404, where a line defined by features 420S and 422S forms a side of triangle 425S. In target image 402, a line defined by features 416T, 418T forms a side of a triangle 424T. Triangle 424T is constructed based on the angles of triangle 424S, such that triangle 424T is similar to triangle 424S, and its third vertex 4283 determines a location in target coordinate system 406, which corresponds to the location of axis mark 4263. Similarly, a line defined by features 420T, 422T forms a side of a triangle 425T, which is similar to triangle 425S. Triangle 425T is constructed based on the angles of triangle 425S, to determine the location in target coordinate system 406 corresponding to axis mark 4266, marked by symbol 4286. The locations in target coordinate system 406 corresponding to axis marks 4261, 4262, 4264 and 4265, marked by symbols 4281, 4282, 4284 and 4285 are similarly determined. Thus, alignment designator symbols 4281-4826 may be drawn at the corresponding locations thereof. More than two anchor points may be employed to determine the conversion of the location of a point in source coordinate system 400 to target coordinate system 406, thus defining multiple triangles. The locations in target coordinate system 406 defined by the plurality of triangles may be averaged. As may be understood from the above, the location conversion method exemplified in FIG. 8 is invariant to relative shift, rotation and scaling between source coordinate system 404 and target coordinate system 406. The location conversion method exemplified in FIG. 8 may be advantageous, for example, when an intraoperative image is distorted, relative to the pre-operative image (e.g., due to blood hemorrhage, liquids or edema occurring during the operation), when the eye gaze direction is not directly towards the camera, or when tools are inserted into the surgical field. In such cases a registration process may be less accurate than the method exemplified in FIG. 8. When the size of the triangles used for converting locations is small relative to the size of the eye, and when multiple triangles are used, this location conversion method exhibits enhanced robustness to the above issues.

When the location of a line such as line 220 as described above FIGS. 2A-2D is to be determined in a second coordinate system, the position of at least two points on the line are determined in the second coordinate system, thereby defining the line.

In general, a location of a line is defined by the locations of a plurality of points located on the line. In some embodiments, to avoid scaling issues, when determining the location of a finite line in the second coordinate system, the locations of the two endpoints of the line are determined in the second coordinate system. The conversion of locations described above in conjunction with FIG. 8 exemplified a conversion of locations employing triangles. However, any geometrical or functional relationship between points that exhibits scale and rotation invariance may be employed to determine the conversion of locations.

Described herein above are examples of conversion of locations between coordinate systems: conversion of locations based on registration of a 3D coordinate system with a tracking coordinate system and tracking a camera, conversion of locations based on image registration and converting selected locations between two images (e.g., without performing registration). It is noted that these examples are brought hereinabove as examples only for conversion of locations. The disclosed technique is applicable regardless of the method of conversion of locations.

It will be appreciated by persons skilled in the art that the disclosed technique is not limited to what has been particularly shown and described hereinabove. Rather the scope of the disclosed technique is defined only by the claims, which follow.

In some embodiments, the technology involves a method for providing visual information relating to a validity of a conversion of locations between a first image and a second image, said first image and second image are images of an eye of a patient employed for ophthalmic surgery, said method including the procedures of selecting at least one image element in said first image, said at least one image element representing a respective physical element, said at least one physical element visible in said second image, said at least one image element associated with at least one location in a first coordinate system associated with said first image. The method also involves determining for said at least one location in said first coordinate system at least one corresponding location in a second coordinate system associated with said second image by employing said conversion of locations between said first coordinate system and said second coordinate system. The method also involves generating a respective verification symbol associated with said at least one image element and superimposing said verification symbol on said second image, based at least on said at least one corresponding location, wherein, when said conversion of locations between said first image and said second image is valid, said at least one physical element visible in said second image and said respective verification symbol appear visually in alignment, and when said conversion of locations between said first image and said second image is invalid, said at least one physical element visible in said second image and respective verification symbol appear visually out of alignment.

In some scenarios, the at least one of said first image and said second images is an intraoperative image, and the other one of said first image and said second images is one of a preoperative image or an intraoperative image.

In some scenarios, the at least one image element is at least one of a scleral blood vessel, a retinal blood vessel, a bifurcation point, a contour of the limbus, and a visible element on the iris.

In some scenarios, guidance information defined with respect to a coordinate system associated with one of the first image or the second image is overlaid on the other one of the first image or the second image employing the conversion of locations. In some scenarios, the guidance information includes at least one of information indicating a planned location and/or orientation of an intraocular lens, the information indicating an actual location and/or orientation of an intraocular lens, the information indicating a planned incision; the information indicating a planned location and/or orientation of an implant (e.g., an implant for treating glaucoma), the information relating to planned sub-retinal injection, the information relating to a membrane removal, the information indicating a location of an OCT scan, and the information indicating a footprint of a field of view of an endoscope.

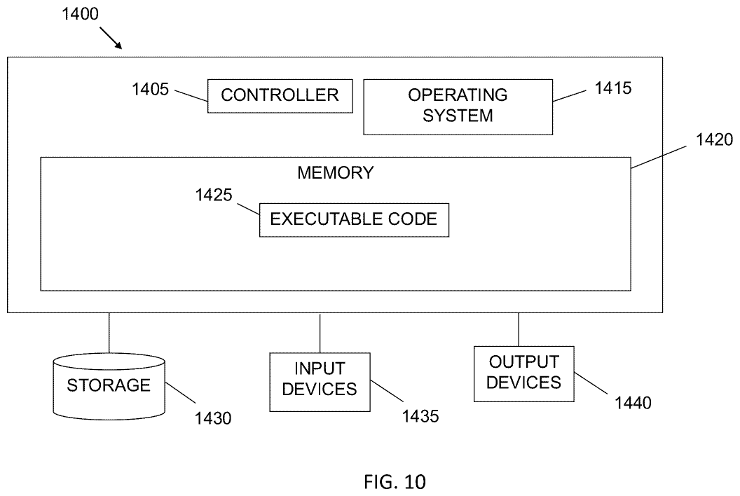

FIG. 10 shows a block diagram of a computing device 1400 which may be used with embodiments of the invention. Computing device 1400 may include a controller or processor 1405 that may be or include, for example, one or more central processing unit processor(s) (CPU), one or more Graphics Processing Unit(s) (GPU or GPGPU), FPGAs, ASICs, combination of processors, video processing units, a chip or any suitable computing or computational device, an operating system 1415, a memory 1420, a storage 1430, input devices 1435 and output devices 1440.

Operating system 1415 may be or may include any code segment designed and/or configured to perform tasks involving coordination, scheduling, arbitration, supervising, controlling or otherwise managing operation of computing device 1400, for example, scheduling execution of programs. Memory 1420 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long term memory unit, or other suitable memory units or storage units. Memory 1420 may be or may include a plurality of, possibly different memory units. Memory 1420 may store for example, instructions to carry out a method (e.g. code 1425), and/or data such as user responses, interruptions, etc.

Executable code 1425 may be any executable code, e.g., an application, a program, a process, task or script. Executable code 1425 may be executed by controller 1405 possibly under control of operating system 1415. For example, executable code 1425 may when executed cause masking of personally identifiable information (PII), according to embodiments of the invention. In some embodiments, more than one computing device 1400 or components of device 1400 may be used for multiple functions described herein. For the various modules and functions described herein, one or more computing devices 1400 or components of computing device 1400 may be used. Devices that include components similar or different to those included in computing device 1400 may be used, and may be connected to a network and used as a system. One or more processor(s) 1405 may be configured to carry out embodiments of the invention by for example executing software or code. Storage 1430 may be or may include, for example, a hard disk drive, a floppy disk drive, a Compact Disk (CD) drive, a CD-Recordable (CD-R) drive, a universal serial bus (USB) device or other suitable removable and/or fixed storage unit. Data such as instructions, code, NN model data, parameters, etc. may be stored in a storage 1430 and may be loaded from storage 1430 into a memory 1420 where it may be processed by controller 1405. In some embodiments, some of the components shown in FIG. 10 may be omitted.

Input devices 1435 may be or may include for example a mouse, a keyboard, a touch screen or pad or any suitable input device. It will be recognized that any suitable number of input devices may be operatively connected to computing device 1400 as shown by block 1435. Output devices 1440 may include one or more displays, speakers and/or any other suitable output devices. It will be recognized that any suitable number of output devices may be operatively connected to computing device 1400 as shown by block 1440. Any applicable input/output (I/O) devices may be connected to computing device 1400, for example, a wired or wireless network interface card (MC), a modem, printer or facsimile machine, a universal serial bus (USB) device or external hard drive may be included in input devices 1435 and/or output devices 1440.

Embodiments of the invention may include one or more article(s) (e.g. memory 1420 or storage 1430) such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein.

Unless specifically stated otherwise, as apparent from the foregoing discussion, it is appreciated that throughout the specification discussions utilizing terms such as “processing,” “computing,” “calculating,” “determining,” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Embodiments of the invention may include an article such as a computer or processor readable non-transitory storage medium, such as for example a memory, a disk drive, or a USB flash memory encoding, including, or storing instructions, e.g., computer-executable instructions, which when executed by a processor or controller, cause the processor or controller to carry out methods disclosed herein.

It should be recognized that embodiments of the invention may solve one or more of the objectives and/or challenges described in the background, and that embodiments of the invention need not meet every one of the above objectives and/or challenges to come within the scope of the present invention. While certain features of the invention have been particularly illustrated and described herein, many modifications, substitutions, changes, and equivalents may occur to those of ordinary skill in the art. It is, therefore, to be understood that the appended claims are intended to cover all such modifications and changes in form and details as fall within the true spirit of the invention.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures, and examples.

It is to be understood that the details set forth herein do not construe a limitation to an application of the invention.

Furthermore, it is to be understood that the invention may be carried out or practiced in various ways and that the invention may be implemented in embodiments other than the ones outlined in the description above. One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the foregoing detailed description, numerous specific details are set forth in order to provide an understanding of the invention. However, it will be understood by those skilled in the art that the invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments.

If the specification or claims refer to “an additional” element, that does not preclude there being more than one of the additional elements.

It is to be understood that where the claims or specification refer to “a” or “an” element, such reference is not to be construed that there is only one of that element.

It is to be understood that where the specification states that a component, feature, structure, or characteristic “may”, “might”, “may” or “can” be included, that a particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined. The present invention may be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method for providing a verification symbol relating to a validity of a conversion of locations between a first image of an eye of a patient and a second image of the eye of the patient, employed for ophthalmic surgery, the method comprising:
receiving a selection of an image element in the first image, the image element corresponding to a physical element visible in the second image, the image element having a location in a first coordinate system, the first coordinate system being associated with the first image;
determining for the location in the first coordinate system a corresponding location in a second coordinate system being associated with the second image by employing the conversion of locations between the first coordinate system and the second coordinate system;
displaying the verification symbol superimposed with the second image based on the corresponding location in the second coordinate system; and
displaying guidance information defined with respect to the first coordinate system superimposed with the second image or guidance information defined with respect to the second coordinate system superimposed with the first image, employing the conversion of locations.

2. The method of claim 1, wherein the selection of the image element is performed either manually or automatically.

3. The method of claim 1, wherein at least one of the first or second images is intraoperative.

4. The method of claim 1, wherein the image element is at least one of:
a scleral blood vessel;
a retinal blood vessel;
a bifurcation of a blood vessel;
a contour of the limbus; and
a visible element in the iris.

5. The method of claim 1, wherein the guidance information comprises at least one of:
information indicating a planned location and/or orientation of an intraocular lens;
information indicating an actual location and/or orientation of an intraocular lens;
information indicating a planned incision;
information indicating a planned location and/or orientation of an implant;
information relating to planned sub-retinal injection;
information relating to a membrane removal;
information indicating a location of an OCT scan; and
information indicating a footprint of a field of view of an endoscope.

6. The method of claim 1, wherein the verification symbol comprises one or more of: a geometrical shape, a color symbol, a differentiating contrast symbol, a variable intensity symbol and a model of at least a portion of the physical element to which the image element corresponds.

7. A system for providing visual information relating to a validity of a conversion of locations between a first image and a second image, the first image and second image employed for ophthalmic surgery, the system comprising:
a camera configured to acquire at least one of the first image and the second image; and
a processor, coupled with the camera configured to:
receive a selection of an image element in the first image, the image element corresponding to a physical element visible in the second image, the image element having a location in a first coordinate system, the first coordinate system being associated with the first image;
determine for the location in the first coordinate system a corresponding location in a second coordinate system being associated with the second image by employing the conversion of locations between the first coordinate system and the second coordinate system; and
display the verification symbol superimposed with the second image based on the corresponding location in the second coordinate system,
wherein the processor is further configured to display guidance information defined with respect to the first coordinate system superimposed with the second image or guidance information defined with respect to the second coordinate system superimposed with the first image, employing the conversion of locations.

8. The system of claim 7, wherein the selection of the image element is performed either manually or automatically.

9. The system of claim 7, wherein at least one of the first or second images is intraoperative.

10. The system of claim 7, wherein the image element is at least one of:
a scleral blood vessel;
a retinal blood vessel;
a bifurcation of a blood vessel;
a contour of the limbus; and
a visible element on the iris.

11. The system of claim 7, wherein the guidance information comprises at least one of:
information indicating a planned location and/or orientation of an intraocular lens;
information indicating an actual location and/or orientation of an intraocular lens;
information indicating a planned incision;
information indicating a planned location and/or orientation of an implant;
information relating to planned sub-retinal injection;
information relating to a membrane removal;
information indicating a location of an OCT scan; and
information indicating a footprint of a field of view of an endoscope.

12. The system of claim 7, wherein the verification symbol comprises one or more of: a geometrical shape, a color symbol, a differentiating contrast symbol, a variable intensity symbol and a model of at least a portion of the physical element to which the image element corresponds.

13. A method for providing a verification symbol relating to a validity of a conversion of locations between a first image and a second image, the first image and the second image employed for ophthalmic surgery, the method comprising:
receiving a first image associated with a first coordinate system;
receiving guidance information with respect to the first image;
receiving a second image associated with a second coordinate system, the second image representing an optical image of a scene;
receiving a selection of an image element in the first image, the image element corresponding to a physical element visible in the optical image, the image element having a first location in the first coordinate system;
determining for the first location in the first coordinate system a corresponding second location in the second coordinate system by employing the conversion of locations between the first coordinate system and the second coordinate system;
determining a third location in the second image of a guidance symbol generated based on the guidance information, by employing the conversion of locations from the first coordinate system to the second coordinate system;
generating an overlay image comprising the guidance symbol and the verification symbol based on the determined third and second locations, respectively; and
displaying the overlay image superimposed with the optical image.

14. The method of claim 13, wherein the guidance information is received as at least one of:
a superimposition with the first image; or separately from the first image.

15. The method of claim 13, wherein the selection of the image element is performed either manually or automatically.

16. The method of claim 13, wherein the image element is at least one of:
a scleral blood vessel;
a retinal blood vessel;
a bifurcation of a blood vessel;
a contour of the limbus; and
a visible element on the iris.

17. A system for providing a verification symbol relating to a validity of a conversion of locations between a first image and a second image, the first image and the second image employed for ophthalmic surgery, the system comprising:
a camera configured to acquire the first image, the second image or both; and
a processor, coupled with the camera, and configured to:
receive a first image associated with a first coordinate system;
receive guidance information with respect to the first image;
receive a second image associated with a second coordinate system, the second image representing an optical image of a scene;
receive a selection of an image element in the first image, the image element corresponding to a physical element in the optical image, the image element having a first location in the first coordinate system;
determine for the first location in the first coordinate system a corresponding second location in the second coordinate system by employing the conversion of locations between the first coordinate system and the second coordinate system;
determine a third location in the second image of a guidance symbol generated based on the guidance information, by employing the conversion of locations from the first coordinate system to the second coordinate system;
generate an overlay image comprising the guidance symbol and the verification symbol based on the determined third and second locations, respectively; and
display the overlay image superimposed with the optical image.

18. The system of claim 17, wherein the selection of the image element is performed either manually or automatically.

19. The system of claim 17, wherein the image element is at least one of:
a scleral blood vessel;
a retinal blood vessel;
a bifurcation of a blood vessel;
a contour of the limbus; and
a visible element on the iris.

20. The system of claim 17, wherein the guidance information is received as a superimposition with the first image, and wherein the guidance information is received separately from the first image.
